(19) United States
(12) Patent Application Publication
(10) Pub. No.: US 2020/0289049 A1
(43) Pub. Date: Sep. 17, 2020

(54) Non-Implantable Medical Devices Integrating A Device For Detecting And/Or Identifying Microbiological Infections

(71) Applicants: Biomerieux, Marcy-L'étoile (FR); Elkem Silicones France SAS, Lyon (FR); F.D.S., Genay (FR); Fibroline, Limonest (FR); Hospices Civils De Lyon, Lyon (FR); Universite Claude Bernard Lyon 1, Villeurbanne (FR); Institut National de la Sante et de la Recherche Medicale (INSERM), Paris (FR); Ecole Normale Superieure De Lyon, Lyon (FR); Centre National de la Recherche Scientifique (CNRS), Paris (FR)

(72) Inventors: Christine Rozand, Saint-Genis-Les-Ollières (FR); Aline Dardy, Lyon (FR); Fanny Berrat, Tassin-La-Demi-Lune (FR); Caroline Moine, Sorbiers (FR); Philippe Serre, Collonges-Au-Mont-D'or (FR)

(73) Assignees: Biomerieux, Marcy-L'étoile (FR); Elkem Silicones France SAS, Lyon (FR); F.D.S., Genay (FR); Fibroline, Limonest (FR); Hospices Civils De Lyon, Lyon (FR); Universite Claude Bernard Lyon 1, Villeurbanne (FR); Institut National de la Sante et de la Recherche Medicale (INSERM), Paris (FR); Ecole Normale Superieure De Lyon, Lyon (FR); Centre National de la Recherche Scientifique (CNRS), Paris (FR)

(21) Appl. No.: 16/767,826

(22) PCT Filed: Nov. 29, 2018
(86) PCT No.: PCT/FR2018/053033

§ 371 Date: May 28, 2020

(30) Foreign Application Priority Data
Nov. 30, 2017 (FR) 1761417

(51) Int. Cl. A61B 5/00; A61F 13/00; A61L 15/24; A61L 15/18; A61F 13/02; A61K 49/00; C12Q 1/14

(52) U.S. Cl. CPC A61B 5/445; A61B 5/6832; A61F 13/00059; A61L 15/24; A61B 5/14539; A61F 13/022; A61K 49/00; C12Q 1/14; A61L 15/18

(57) ABSTRACT

The present invention relates to a non-implantable medical device (1, 1′) intended to cover wounds and skin lesions, comprising a biosensor (3E, 3E′), characterised in that said biosensor (3E, 3E′) comprises a piece made of absorbent, hydrophilic material fixing, on the surface and/or within the thickness thereof, a composition of agglomerated powders comprising particles of ethylene vinyl acetate (EVA) having a surface in part covered with at least one orthophosphoric acid salt and a visual indicator of microbiological growth.

In particular, the medical device (1, 1′) may comprise several layers in order to enable the guiding of a liquid.

The present invention relates to a method for preparing such a medical device (1, 1′).

The present invention belongs to the field of non-implantable medical devices used to cover wounds and skin lesions with a view to protecting them from soiling and external infections, such as dressings, compresses, bandages and other analogous articles. The present invention more precisely pertains to non-implantable medical devices integrating means for diagnosing microbiological infections with a view to detecting the occurrence of a potential infection.

Wounds, whatever their seriousness, are subjected to risks of infection by pathogenic microorganisms such as bacteria, viruses, fungi, protozoa. Apart from the fact of interrupting the physiological process of healing, these infections may lead to serious complications, such as gaseous gangrene, tetanus and long term incapacities (chronic infection of the wound or the bone, etc.), or even to death consecutive to sepsis.

In the case of hospitalisations, more than 90% of post-operative infections observed occur within the 30 days following surgical intervention. Patients must thus be monitored at least for the 30 days after having been operated. Clinically, this monitoring generally consists of a visual inspection of the healing of wounds and the observation of a potential purulent discharge, which—depending on the colour, the consistency and the smell-may be the sign of an infection. When an infection is suspected, a sampling is carried out then dispatched to the analytical laboratory for complementary investigations: microscopic analyses, microbiological cultures and/or antibiograms, etc. In practice, the practitioner will only have at his disposal the results of analyses the next day, at the earliest.

For better monitoring of patients, health professionals (physicians, practitioners, nurses, etc.) have expressed the desire to be able to have at their disposal diagnostic tests and tools allowing them to observe by themselves the existence of an infection and, optionally, to envisage the treatment to prescribe during the actual consultation.

In this context, it has been envisaged to integrate means for detecting and/or diagnosing microbiological infections in the structure of compresses, dressings, bandages and other non-implantable medical devices used to cover and to protect wounds.

In a first approach, on the basis of recent technological advances in the field of microelectrodes, electrochemical sensors have been developed to carry out in situ electrochemical analyses of the chemical composition of the exudates of wounds and to do so with a view to detecting the presence of biomarkers associated with microbiological infections.

In this context, it is possible to cite notably the works of Hajnsek et al., 2015 (SENSOR AND ACTUATORS B: CHEMICAL; 265-274: “An electrochemical sensor for fast detection of wound infection based on myeloperoxidase activity”) and Sismaet et al., 2016 (WOUND REPAIR AND REGENERATION; (24)366-372: “Electrochemical detection of Pseudomonas in wound exudate samples from patients with chronic wounds”). In the first example, the sensor enables the electrochemical detection of myeloperoxidase activity, which is expressed by numerous pathogenic bacteria. In the second example, the sensor enables electrochemical detection of pyocyanin, a molecule produced by Pseudomonas aeruginosa.

These electrochemical sensors are today at an early experimental stage of their development. They embed a technology of miniaturised electrodes of which the current cost is incompatible with the market for dressings and compresses. Furthermore, such electrochemical sensors are not autonomous. To operate, they must be connected to an external item of equipment capable of supplying them with electricity, collecting the data transmitted by the sensors, and processing said data before being able to return an analysis result to the practitioner; as many items of equipment that said practitioner will need to have at his disposal.

In a second approach, more in keeping with the technological and economic constraints of the market for dressings and compresses, the idea of a smart bandage has been conceived. Placed on the wound, this would generate a visible signal (appearance of a colour and/or fluorescence) in the event of occurrence of a microbiological infection.

In this context, Brocklesby et al., 2013 (Medical hypotheses; (80)237-240: “Smart bandages—A colourful approach to early stage infection detection & control the wound care”) envisage the production of a dressing enabling the detection of bacteria producing β-lactamases. To do so, it is suggested to use a “chimeric” chromogenic cephalosporin, assembled from a residue of cephalosporin and a chromophore. This “chimeric” chromogenic cephalosporin would be fixed by means of chemical coupling to the fibres of the absorbent textile constituting the dressing. Its hydrolysis by cephalosporinases would cause coloration of the dressing. To date, no device or dressing operating on this detection principle has seen the light of day.

More recently, researchers from the University of Bath in the United Kingdom have announced the development of an intelligent dressing prototype emitting fluorescence in response to bacterial infection (Thet et al., 2016—ACS Applied Materials & Interfaces; 8(24): 14909-19: “Prototype development of the intelligent hydrogel wound dressing and its efficacy in the detection of model pathogenic wound biofilm”).

This dressing is formed of a piece of absorbent tissue, of which the face intended to be applied in actual contact with the wound of the patient is covered with a 2% agarose hydrogel. In the bulk of this hydrogel are distributed vesicles loaded with 5,6-carbofluresceine. The wall of these vesicles is constituted of an assembly of phospholipids, cholesterol and polyacetylene polymers. By its composition, it mimics the phospholipidic membrane of eukaryotic cells and is perforated by the cytotoxins that the pathogenic bacteria release in the course of an infection. 5,6-carbofluresceine, for its part, is a compound which has two interesting particularities, ingeniously exploited in this device. The first is that it self-assembles into dimers, when its concentration is sufficiently high, then dissociates as its concentration decreases. The second particularity is that it emits fluorescence when it is in the monomeric state; in the dimer state, its fluorescence is on the other hand blocked.

In the event of bacterial infection, the wall of the vesicles is broken by the released cytotoxins, and 5,6-carbofluresceine is freed from the vesicles and is diluted progressively in the hydrogel. Exposed to UV light, the dressing emits fluorescence.

At present, this device is at the prototype stage and numerous tests are planned to ensure the innocuity of the agarose hydrogel, of that of the vesicles and of 5,6-carbofluresceine, which will be placed directly in contact with the wound of the patient.

Such a device has several major drawbacks. A first drawback is linked to the non-specificity of detection. Such a device will only be able to alert the practitioner of the occurrence of an infection. Complementary tests will have to be carried out, if the practitioner wishes to know the nature of the incriminated pathogen(s) and to envisage the prescription of a specific treatment. A second drawback stems from the stability over time of the agarose hydrogel and pertains more to considerations specific to the industrial and commercial exploitation of such a device. Due to the presence of this hydrogel, the commercial success of such a device will suffer from a short shelf life and important packaging and conservation constraints.

The present invention aims to resolve the aforementioned drawbacks. It thus has for general aim to propose a technical solution to the production of so-called smart dressings, notably in terms of simplicity of implementation and specificity of detection.

An objective of the present invention is to propose a non-implantable medical device intended to cover wounds and skin lesions, of dressing, compress, bandage type and other absorbent articles, integrating means for diagnosing microbiological infections and making it possible to signal a state of bacterial infection by a change of colour and/or by the emission of fluorescence. Another objective of the present invention is to enable detection and specific identification of the pathogen(s) in question, thanks to the appearance of a coloration profile and/or emission of fluorescence, particular and characterizable.

The ambition of the present invention is to propose non-implantable medical devices intended to cover wounds and skin lesions, of dressing, compress, bandage type, enabling the in situ diagnosis of microbiological infections, and to do so in a manner compatible with the constraints of an industrial and commercial exploitation specific to this sector.

Finally, the aim of the present invention is to propose a technology that can be exploited not just for human medicine but also for veterinary medicine.

In this context, the object of the present invention is a non-implantable medical device intended to cover wounds and skin lesions, comprising a biosensor, characterised in that said biosensor comprises a piece made of absorbent, hydrophilic material fixing, on the surface and/or within the thickness thereof, a composition of agglomerated powders comprising particles of ethylene vinyl acetate (EVA) having a surface in part covered with at least one orthophosphoric acid salt and a visual indicator of microbiological growth.

The non-implantable medical device is used to cover wounds and skin lesions with a view to protecting them from soiling and external infections, and may be a dressing, a compress, a bandage and any other absorbent article suited to medical use.

The device according to the invention makes it possible, due to the presence of the biosensor, to diagnose a microbiological infection in situ.

In order to enhance detection, the device according to the invention could comprise means making it possible to direct blood or exudate from a wound to the biosensor.

As defined in the present invention, the term “liquid” will be used to designate blood or exudate from a wound or at least one constituent element of said exudate or more generically any aqueous biological liquid (or “biological fluid” or “body fluid”) capable of containing infectious microorganisms.

Thus, in a preferred embodiment, the non-implantable medical device further comprises a primary layer suited to directing a liquid to the biosensor. Said primary layer advantageously has a preferential collection zone of said liquid as well as means for guiding said liquid in the direction of said preferential collection zone.

Advantageously, the non-implantable medical device further comprises a secondary layer integrating said biosensor, the secondary layer being arranged with respect to the primary layer so that said biosensor extends facing the preferential collection zone. The secondary layer advantageously comprises first and second zones, said first and second zones being distinct and each having a different respective behaviour in contact with said liquid.

Advantageously, the non-implantable medical device further comprises an intermediate layer, which is interposed between said primary and secondary layers. Advantageously, the intermediate layer itself comprises at least one first portion impermeable to said liquid and at least one first permeable portion enabling the passage of said liquid coming from the primary layer to the secondary layer, said first impermeable portion extending facing said first zone, the projection of the first impermeable portion in a plane parallel to said secondary and intermediate layers covering at least half of the surface of the projection of the first zone in this same plane, to limit reflux of said liquid from said first zone to said primary layer.

Advantageously, the non-implantable medical device further comprises a barrier layer, covering said secondary layer and being substantially impermeable to said liquid. Said barrier layer is advantageously permeable to air, and preferably transparent to light.

Advantageously, the non-implantable medical device further comprises a transfer layer permeable to said liquid, arranged facing said primary layer and enabling the passage of said liquid coming from the medium external to said medical device to said primary layer.

Such a multilayer medical device makes it possible to limit reflux of liquid through the different layers of said medical device, while being of extremely simple, compact and reliable design.

A multilayer medical device is an assembly advantageously obtained by stacking a plurality of surface layers distinct from each other, these layers preferably extending along respective planes substantially parallel to each other. As will be described in greater detail hereafter, the different layers which compose such a medical device are preferentially distinct and of different nature and function, such that said medical device is advantageously composite.

The medical device is advantageously intended to guide a given liquid and designed to modify the position of at least one part of said liquid with which said medical device is placed in contact. More specifically, the multilayer medical device is advantageously designed to displace, convey, a certain quantity of said liquid from at least one first region of said medical device to at least one second region of the latter, said second region being preferably distant and distinct from said first region and said second region integrating the biosensor. Thus, as will be made explicit hereafter, the different layers forming the multilayer medical device of the invention are preferentially designed, configured and laid out with respect to each other to enable a displacement of the considered liquid from said first region to said second region of the medical device, said first and second regions advantageously belonging to two distinct layers not immediately adjacent. The displacement of said liquid comprises, preferably, at least one component orthogonal to the layers, or even advantageously a combination of at least one component parallel to the layers (that is to say a displacement in the plane of extension of at least one of the layers of the medical device) and at least one component orthogonal to said layers. In this sense, the multilayer medical device of the invention advantageously constitutes a fluidic system or at least a fluidic system component.

The medical device of the invention is advantageously a dressing or a compress, more advantageously a dressing.

Other aims, characteristics and advantages of the invention will become clear in view of the description that follows and the examples developed hereafter, which refer to the appended figures and in which:

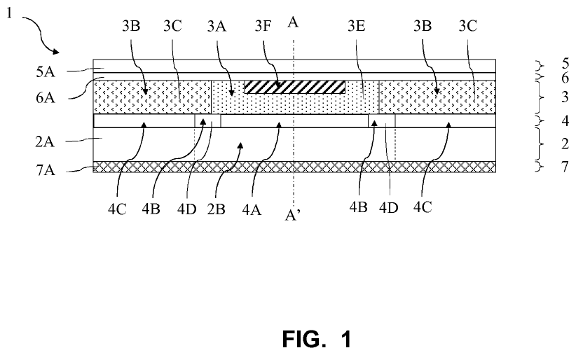

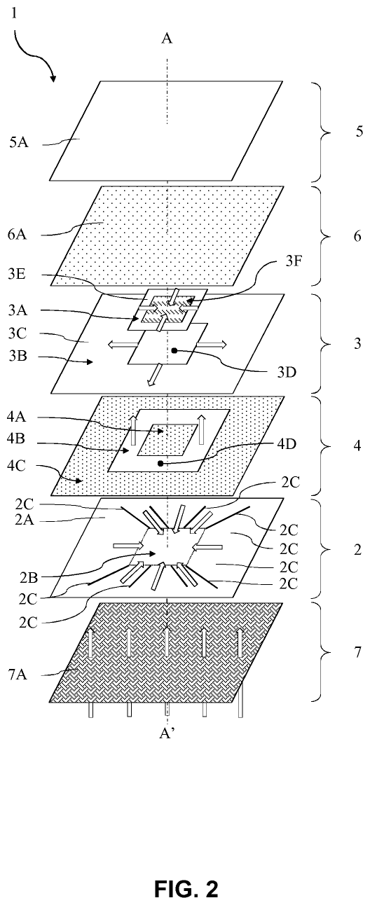

FIGS. 1 and 2 illustrate, schematically, and respectively according to a vertical section and according to a perspective view, a preferential embodiment of the multilayer medical device of the invention;

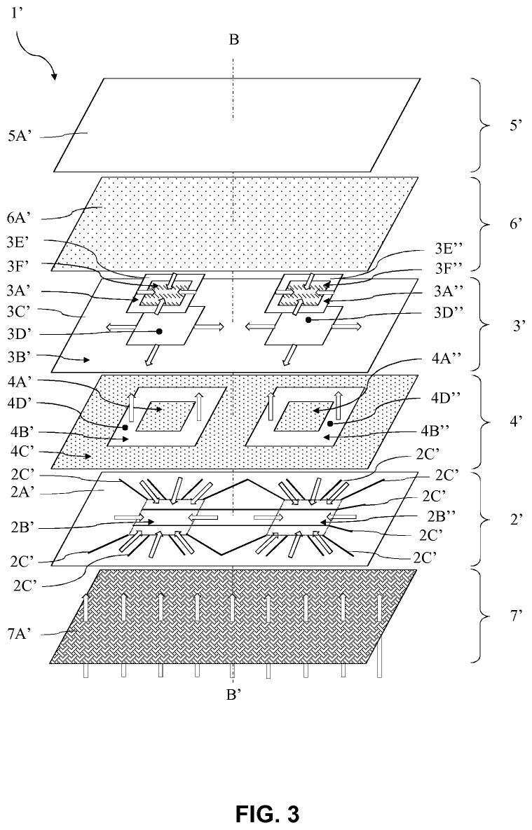

FIG. 3 illustrates, schematically and in perspective, another preferential embodiment of the multilayer medical device of the invention;

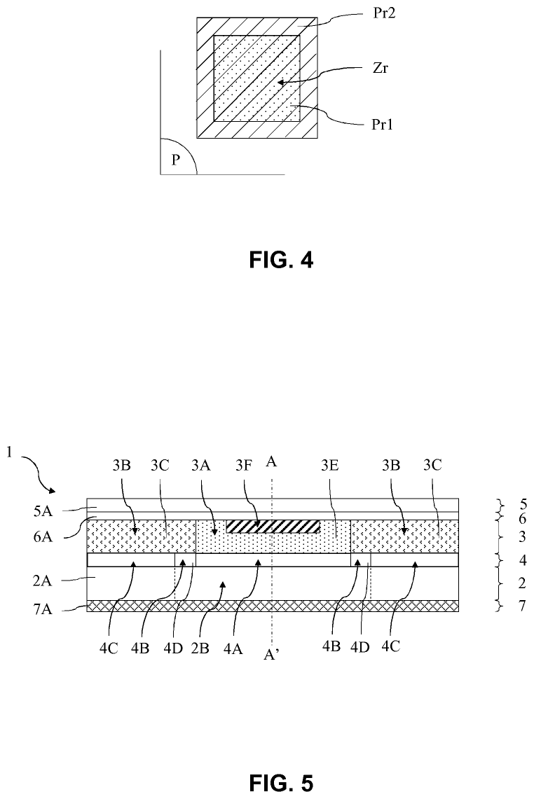

FIG. 4 illustrates, schematically and in top view, the covering of the respective orthogonal projections in a same fictive plane of certain elements of the layers of the multilayer medical device of the invention;

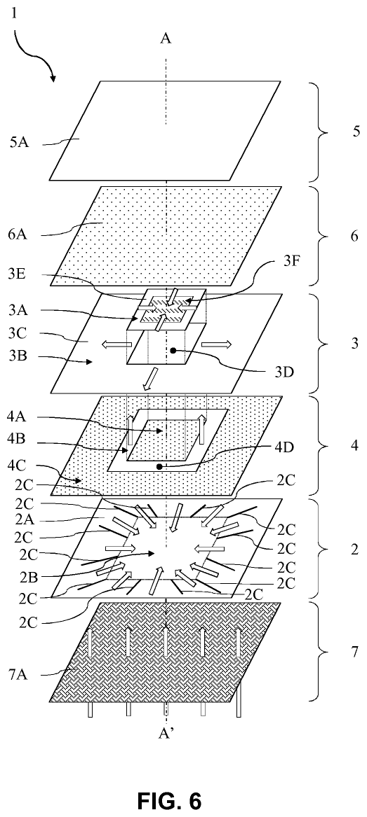

FIGS. 5 and 6 illustrate schematically, and respectively according to a vertical section and according to a perspective view, an alternative of the preferential embodiment of FIGS. 1 and 2;

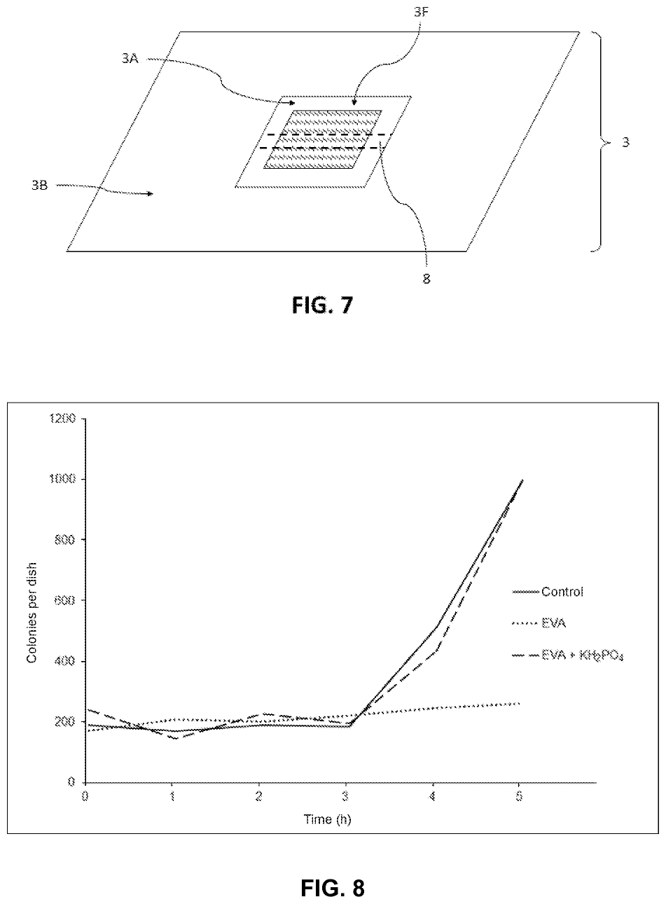

FIG. 7 illustrates, schematically and in perspective, an alternative of the secondary layer of the medical device according to the invention, which has a compression line;

FIG. 8 is a graphic representation of the bacteriostatic effect of EVA on the development of bacteria, and the inhibition of this bacteriostatic effect by orthophosphoric acid salts;

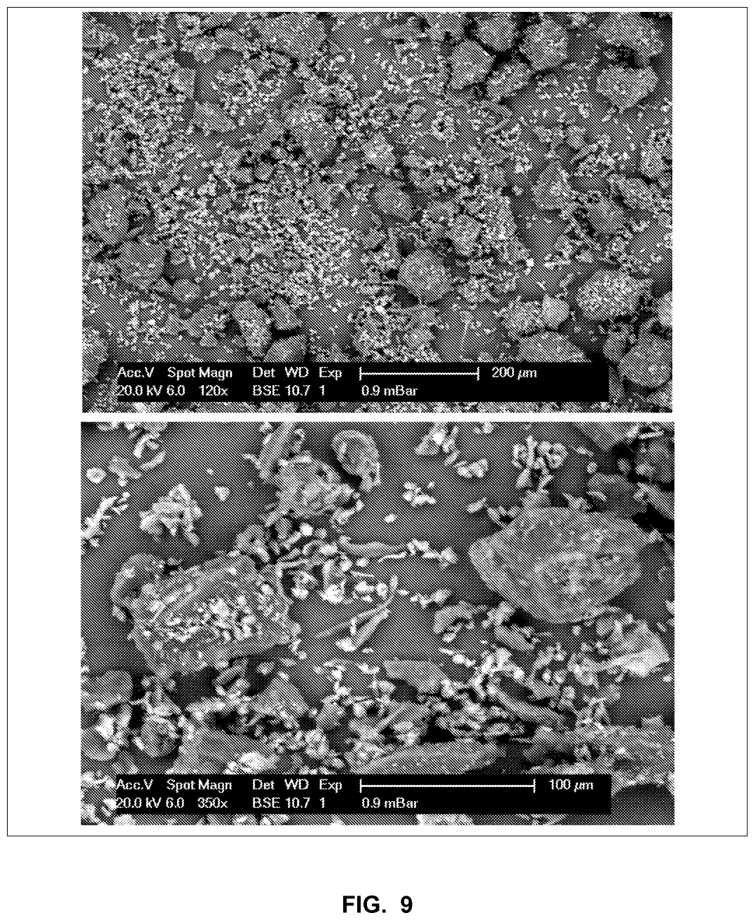

FIG. 9 corresponds to two photographs, taken by scanning electron microscope, showing a powdery composition of dissociated particles;

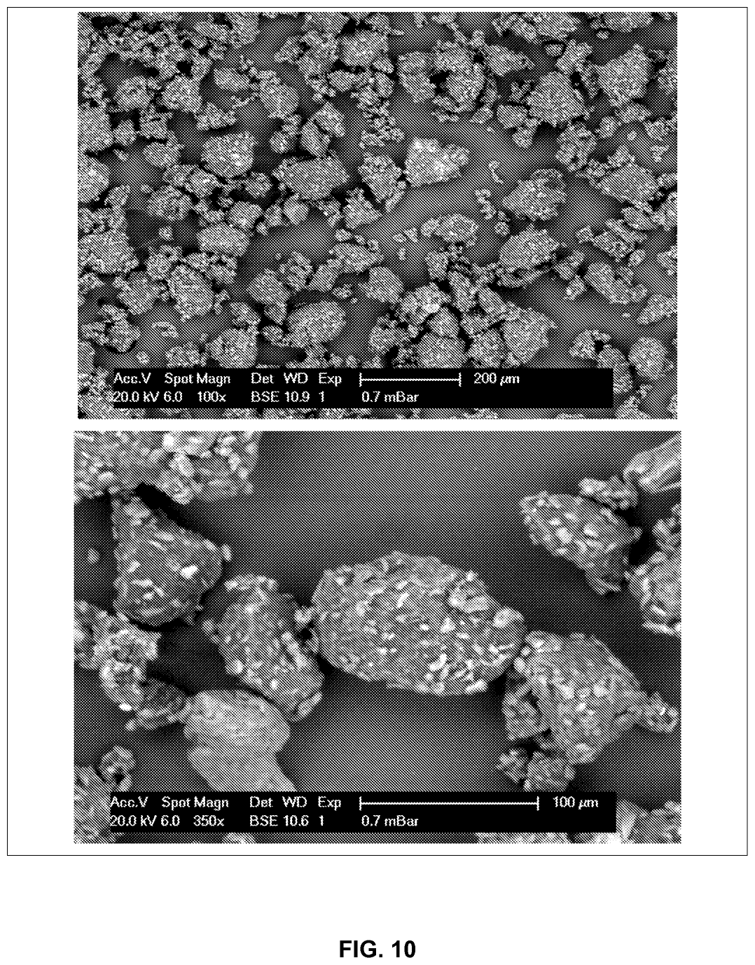

FIG. 10 corresponds to two photographs, taken by scanning electron microscope, showing a composition of agglomerated powders comprising particles of EVA having a surface in part covered with at least one orthophosphoric acid salt and a visual indicator of microbial growth.

Biosensor

The biosensor comprises a piece made of absorbent, hydrophilic material fixing, on the surface and/or within the thickness thereof, a composition of agglomerated powders comprising particles of ethylene vinyl acetate (EVA) having a surface in part covered with at least one orthophosphoric acid salt and a visual indicator of microbiological growth. In the presence of microorganisms, in particular bacteria and yeasts, said visual indicator of microbiological growth is capable of producing a signal visible to the naked eye when the biosensor is exposed to white light and/or to UV radiation.

Positioned near to a wound or lesion, the medical device is designed to be able to capture and collect exudate from the wound, to guide it to the biosensor and to form a support suited to the growth and to the development of microorganisms. The detection and/or the identification of microorganisms optionally present is carried out thanks to visual indicators (chromogenic and/or fluorogenic) of microbiological growth, sensitive to the metabolic activity of the searched for microorganisms, present in the biosensor.

To design such a biosensor, the inventors have taken advantage of in vitro microbiology techniques targeting the detection and identification of microorganisms such as bacteria, yeasts, moulds and amoeba, and have more particularly been inspired by the works carried out from the 1990s on chromogenic and fluorogenic culture media (Perry et al., 2007—Journal of Applied Microbiology; 103:2046-55: “The application of chromogenic media in clinical microbiology”).

From the composition of these culture media, the inventors have essentially taken up and applied the teachings dispensed on visual indicators of microbiological growth, the use of which makes it possible to visually highlight microorganisms via the metabolic activity thereof. In the literature and in the market are found numerous visual indicators of microbiological growth, capable of being able to be used to make a biosensor according to the invention. They may be, for example, coloured pH indicators, sensitive to the variation in pH induced by hydrolysis of sugars or other substrates metabolizable by the target microorganisms. More preferentially, they are chromogenic and/or fluorogenic substrates capable of releasing a chromophore and/or fluorophore compound under the action of a particular enzymatic activity (such as for example, the activities β-glucuronidase, β-galactosidase, β-glucosidase, α-glucuronidase, α-galactosidase, α-glucosidase, esterase, nitroreductase, phosphatases, aminopeptidase, etc.).

To eliminate any risk of secondary infection of the wound which could be imputable to the medical device, notably to the biosensor, the inventors have taken care, in the design of the latter, to banish any ingredient having nutritive potential with regard to microorganisms. Advantageously, the composition of agglomerated powders fixed in the biosensor of the medical device according to the invention has no nutritive quality notable/measurable for the growth and the development of microorganisms. In particular, this composition of agglomerated powders is exempt of any physiologically effective quantity of carbohydrates, lipids, amino acids and growth factors, provided in the form of chemically well-defined compositions or in the form of complex compositions such as mixtures of peptones, yeast extracts, serums, etc.

Thus, with a medical device according to the invention, in the case of microbiological infection of the wounds, the microorganisms can only tap the nutrients necessary for their development from the exudate of the wound, the only nutritive source available in this particular environment.

To embed and immobilise in the absorbent material of the biosensor a quantity of visual indicator of microbiological growth which is operative for detection and/or identification of the target microorganisms, it was necessary to look for an adhesive being able to meet the following three major conditions:
to be capable of efficiently fixing the visual indicators of microbiological growth to the absorbent, hydrophilic material, without adversely affecting their operation nor hampering their hydrolysis in the event of infection by the target microorganisms,
to be sufficiently efficient so that the quantity to use is not detrimental to the absorption capacities of the absorbent, hydrophilic material,
to be physiologically neutral with regard to microorganisms, that is to say not exhibit any notable activator or inhibitor effects on the growth and the development of microorganisms.

None of the adhesive materials tested by the inventors was able to fully satisfy these three conditions. However, among the numerous materials tested, EVA generated a great amount of interest for the inventors.

A synthetic resin well known since the 1950s for its thermoplastic and thermosetting properties, EVA is prepared by copolymerisation of ethylene with vinyl acetate (the content by weight of vinyl acetate generally varies between 10 and 40%). It is found in very diverse forms in numerous sectors of industry (for example, a plastic film, an adhesive for heat sealing of packages, an adhesive in the binding of books, a superficial layer of the glazed paper of magazines, a plastifier in varnish and paint compositions, a component of a thermomoulded plastic material, a component of a composition for encapsulation of active ingredients, etc.). Within the context of the development of biosensors of medical devices according to the invention, the inventors have been able to observe a handling and a use of EVA of great simplicity. Heating to temperatures of the order of 50-60° C. makes it possible to soften these particles and to make their surface sticky. Heated to such temperatures, the particles of EVA have good surface adherence for a large number of compounds, in particular for the particles of visual indicators of microbiological growth that they can fix and expose on their surface. Unfortunately, the inventors have also observed a bacteriostatic activity of EVA, making it unsuitable for an application in the field of microbiology and all the more so when the final purpose is a cellular culture for the purposes of detection and/or identification. However, through persistence, the inventors have managed to overcome this obstacle and have demonstrated that the addition of an orthophosphoric acid salt makes it possible to inhibit this undesirable bacteriostatic effect.

Advantageously and according to the invention, the orthophosphoric acid salt(s) used to inhibit the bacteriostatic effect of EVA is (are) chosen from potassium dihydrogen phosphate (KH₂PO₄) and sodium dihydrogen phosphate (NaH₂PO₄).

According to a first embodiment, potassium dihydrogen phosphate is used to inhibit the bacteriostatic effect of EVA. The KH₂PO₄/EVA ratio by weight is comprised between 1:25 and 1:8, preferentially comprised between 1:16 and 1:12.

According to a second embodiment, sodium dihydrogen phosphate is used to inhibit the bacteriostatic effect of EVA. The NaH₂PO₄/EVA ratio by weight is comprised between 1:15 and 1:3, preferentially comprised between 1:10 and 1:5.

According to a third embodiment, to inhibit the bacteriostatic effect of EVA, KH₂PO₄ and NH₂PO₄ are used concomitantly. Thus, KH₂PO₄ and NaH₂PO₄ are concomitantly present on the surface of the particles of EVA.

Advantageously and according to the invention, the EVA has a content by weight of vinyl acetate of 10 to 40%, preferentially of the order of 20 to 35%.

Advantageously and according to the invention, the surface of the particles of EVA is also covered in part with a gelling agent which, after absorption of the exudate, gives a gel. Using a gelling agent in this particular context has several interests. Firstly, it improves the absorption performance of the piece made of absorbent, hydrophilic material, constituting the biosensor. Also, it makes it possible to retain and block in position the captured microorganisms.

Numerous gelling agents may be used for this purpose, notably agar, agarose, guar gum and xanthan gum. Xanthan gum is preferred.

According to a particular embodiment of a biosensor of the medical device according to the invention, the surface of the particles of EVA may also be in part covered with a selective agent. This selective agent is chosen to block or slow down the development of the appended flora, rather than that of a target-microorganism or a target-group of microorganisms. It essentially involves compounds with antibiotic and/or antifungicidal effects, having a certain specificity of toxicity (lower toxicity for the target-microorganisms than for the appended flora).

According to a particular embodiment of a biosensor of the medical device according to the invention, the surface of the particles of EVA may also be in part covered with an agent for stimulating the metabolism of bacteria, for example manganese chloride (MnCl₂).

Advantageously and according to the invention, the particles of EVA have a particle size comprised between 40 and 150 μm. The particles of orthophosphoric acid salt(s), the particles of visual indicator(s) of microbiological growth, optionally the particles of gelling agent, the particles of agent for stimulating the metabolism of bacteria and the particles of selective agent, which cover the surface of these particles of EVA, have a particle size advantageously comprised between 5 and 50 μm.

As regards the piece made of absorbent, hydrophilic material constituting a biosensor, numerous materials may be used. Advantageously a fibrous material is used, composed of non-woven fibres, forming an assembly having an integrity and a mechanical coherence. Said fibres may be natural cellulose fibres (such as cotton) or synthetic fibres (such as rayon), modified cellulose fibres (for example, nitrocellulose), fibres of chemical polymers (such as acrylate/acrylamide copolymers).

Advantageously, said piece made of absorbent material has a grammage comprised between 50 g·m⁻² and 200 g·m⁻², for a thickness advantageously comprised between 0.5 mm and 4 mm.

According to a preferred embodiment, this piece made of absorbent, hydrophilic material is made from a non-woven textile, for example based on cellulose fibres. According to this preferred embodiment, the particles of EVA are advantageously fixed to the fibres of the material, essentially within the thickness thereof.

Primary Layer

The medical device advantageously further comprises a primary layer suited to directing a liquid to the biosensor.

This primary layer thus makes it possible to direct a liquid to at least one precise point of the medical device where a biosensor will have been positioned for this purpose, advantageously to direct said liquid in sufficient quantity and in the most rapid and precise manner possible to at least one precise point of the medical device where a biosensor will have been positioned for this purpose.

The primary layer of the medical device enables the guiding of a liquid making it possible to collect exudates from the wound or at least one constituent element of said exudates and to convey them rapidly and with precision to the immediate proximity of the biosensor to enable diagnosis and to signal a state of bacterial infection by a change of colour and/or by the emission of fluorescence.

The primary layer makes it possible to direct the liquid to analyse in sufficient quantity and in the most rapid and precise manner possible to at least one precise point of the medical device where a biosensor will have been positioned for this purpose. Thus, this primary layer makes it possible to concentrate the collected liquid at the level of the wound at at least one very precise point of the medical device.

The primary layer enables a rapid and precise conveyance of said liquid to a predefined precise target zone of the latter or to a plurality of predefined precise target zones of the latter, distinct and distant, while being of extremely simple, compact and reliable design.

The primary layer further has a particularly efficient anti-reflux effect.

The primary layer is particularly easy to design and manufacture using conventional technical means.

According to the invention, the medical device 1, 1′ advantageously comprises at least one primary layer 2, 2′ intended to come into contact with a liquid. Preferably, this primary layer 2, 2′ is intended to come primarily into contact with said liquid, that is to say that it advantageously constitutes a layer of said medical device 1, 1′ which is intended to come into contact with the liquid before all or part of the other layers of said medical device 1, 1′, said liquid thus advantageously penetrating said medical device 1, 1′, from the external environment to the latter, through said primary layer 2, 2′. Preferably, said primary layer 2, 2′ constitutes one of the most superficial layers of the medical device 1, 1′. This primary layer 2, 2′ could thus advantageously correspond to said first region of the medical device 1, 1′ mentioned above.

Preferably, this primary layer 2, 2′ is formed of a primary piece 2A, 2A′ of textile, which is preferentially non-woven, porous and/or hydrophilic (for example made of paper or cotton). Said primary piece 2A, 2A′ of textile is advantageously draining, that is to say that if said primary piece 2A, 2A′ may optionally absorb said liquid on account of the porous and/or hydrophilic character thereof, it is however advantageously substantially exempt of any property of retention of said liquid and it is, conversely, preferentially provided with a large capacity of diffusion of said liquid therewithin. Further preferably, the thickness of the primary layer 2, 2′ is substantially comprised between 0.1 and 0.5 mm.

Advantageously, said primary layer 2, 2′ comprises at least one preferential collection zone 2B, 2B′, 2B″ (identified in dotted lines in FIGS. 2, 3 and 6) of said liquid, as well as means for guiding 2C, 2C′ said liquid in the direction of said collection zone 2B, 2B′, 2B″. Said preferential collection zone 2B, 2B′, 2B″ thus advantageously corresponds to a particular predefined zone of said primary layer 2, 2′ to which said liquid penetrating into the medical device 1, 1′ is preferentially directed, advantageously according to a displacement of said liquid parallel to the primary plane of extension of said primary layer 2, 2′. Preferably, as illustrated in the figures, said preferential collection zone 2B, 2B′, 2B″ is substantially localised at a distance from the primary edges which define the primary contour of said primary layer 2, 2′, so as to limit the risk of leakage of said liquid out of the primary layer 2, 2′ by the primary edges of the latter. Advantageously, the primary layer 2, 2′ having a first surface area S1 (area), the preferential collection zone 2B, 2B′, 2B″ has for its part a second surface area S2 which is substantially less than said first surface area S1 of the primary layer 2, 2′. Thus, said preferential collection zone 2B, 2B′, 2B″ could be defined such that said second surface area S2 is preferentially equal to or less than one half, one third, one quarter, one sixth or even one tenth of said first surface area S1 of the primary layer 2, 2′, depending notably on the targeted application and the quantity of said liquid that the fluidic device is intended to handle.

It should be noted that, if said preferential collection zone 2B, 2B′, 2B″ is represented in the figures according to a rectangular shape, this shape is purely schematic and is given uniquely for illustrative purposes, and could obviously take any other desired shape (circular, oblong, etc.).

Preferably elongated, and preferably substantially rectilinear and continuous, said guiding means 2C, 2C′ are distinct from each other, that is to say that they are arranged at a distance from each other and do not touch or overlap. Moreover, and in a particularly interesting manner, said guiding means 2C, 2C′ are oriented along (mean) directions of longitudinal extension secant at a point (fictive, not represented) situated in (or at least in the immediate proximity of) said preferential collection zone 2B, 2B′, 2B″, to define between them at least one (and preferably a plurality of) primary channel (represented by an arrow in the figures) for guiding said liquid which converges in the direction of said preferential collection zone 2B, 2B′, 2B″.

Preferably, said guiding means 2C, 2C′ are laid out around the preferential collection zone 2B, 2B′, 2B″. Preferentially, and as is particularly visible in FIGS. 2, 3 and 6, the barrier elements 2C, 2C′ are laid out to form two by two convergent “channels” which direct (that is to say “force” the displacement) said liquid present in the primary layer 2, 2′ to said preferential collection zone 2B, 2B′, 2B″. Preferably, said guiding means 2C, 2C′ (in this case, preferably, said barrier elements 2C, 2C′) are laid out around the preferential collection zone 2B, 2B′, 2B″. Further preferably, they are laid out substantially over the whole contour of the preferential collection zone 2B, 2B′, 2B″, so as to surround the latter. Advantageously positioned with respect to each other in a substantially regular manner, or even evenly distributed, around the preferential collection zone 2B, 2B′, 2B″, the guiding means 2C, 2C′ thus preferably define a “star” or “ray” pattern substantially centred on the preferential collection zone 2B, 2B′, 2B″.

Advantageously, the respective primary proximal end of each guiding means 2C, 2C′ is in contact with the primary contour of said preferential collection zone 2B, 2B′, 2B″, said guiding means 2C, 2C′ not extending, preferably, within the preferential collection zone 2B, 2B′, 2B″ itself.

It has been observed that such a configuration of the guiding means 2C, 2C′ and of the preferential collection zone 2B, 2B′, 2B″ makes it possible to create a phenomenon of particularly efficient guided drainage of the liquid in contact with said primary layer 2, 2′ in the direction of said preferential collection zone 2B, 2B′, 2B″, along a direction substantially parallel to the first mean plane P1 of extension of the blanket 2. Moreover, such a configuration also proves to be particularly efficient for very considerably limiting any risk of reflux of said liquid from the preferential collection zone 2B, 2B′, 2B″ to the peripheral edge(s).

Said guiding means 2C, 2C′ are arranged in at least one part of the thickness, and preferably throughout the thickness, of said primary layer 2, 2′. In this way, said at least one primary channel is advantageously defined between two guiding means 2C, 2C′ and throughout the thickness of the primary layer 2, 2′. Any risk of leakage and diffusion, within the thickness of the primary layer 2, 2′, of the liquid circulating in a primary channel is thus avoided.

To guarantee the most efficient possible guiding of said liquid, said guiding means 2C, 2C′ are preferably substantially impermeable to said liquid, and thus each constitute a substantially leak tight obstacle opposing the passage of said liquid therethrough. Such impermeability may be obtained in a physical manner (guiding means 2C, 2C′ made of a non-porous material facing the considered liquid) or chemical manner (guiding means 2C, 2C′ made of a material for example insoluble in said considered liquid).

Said guiding means 2C, 2C′ are preferably made of hydrophobic polymer material, and further preferably non-porous vis-à-vis the liquid. Such a hydrophobic polymer material may, for example, be chosen in the group comprising: a silicone, a polyurethane, a polyethylene, a polyamide and a polyester. Advantageously, said hydrophobic polymer material could be a thermoplastic polymer material, preferably a hot melt adhesive of pressure sensitive adhesive (PSA) type.

As will be explained hereafter, such guiding means 2C, 2C′ may, for example, be produced by local coating and impregnation of said primary piece 2A, 2A′ of textile using said hydrophobic polymer material. Obviously, said guiding means 2C, 2C′ could be constituted in a different manner, without however going beyond the scope of the invention.

According to a first preferential embodiment illustrated in FIGS. 1, 2, 5 and 6, the primary layer 2 comprises a single preferential collection zone 2B. However, said primary layer 2′ could comprise several distinct preferential collection zones. Thus, according to a second preferential embodiment, illustrated in FIG. 3, the primary layer 2′ preferentially comprises first 2B′ and second 2B″ preferential collection zones, distinct and preferentially distant from each other. Advantageously, said guiding means 2C′ will then be laid out according to a plurality of “star” or “ray” patterns respectively centred on each of said first 2B′ and second 2B″ preferential collection zones. Moreover, in order to favour a substantially simultaneous and homogeneous guiding of said liquid in the direction of each of the preferential collection zones 2B′, 2B″, the primary layer 2′ could advantageously comprise a plurality of additional barriers for guiding said liquid, laid out in a substantially parallel manner and defining at least one additional channel for guiding said liquid which connects between them said preferential collection zones 2B′, 2B″ (FIG. 3).

Advantageously, as illustrated in FIG. 3, said preferential collection zone 2B′ and said preferential collection zone 2B″ are advantageously identical to each other and positioned mirror-wise on either side of the plane orthogonal to the mean plane of extension of the primary layer 2′. The preferential collection zone 2B′ and the guiding means 2C′ define a “fluidic pattern” which is advantageously reproduced identically by symmetry. Alternatively, it could obviously be possible to envisage, without however going beyond the scope of the invention, that the preferential collection zone 2B′ has a shape and/or dimensions entirely different from that or those of the preferential collection zone 2B″, and/or that the conformation and the configuration of the guiding means 2C′ differ from one fluidic pattern to the next.

In order to favour a substantially simultaneous and homogeneous guiding of said liquid in the direction of each of said preferential collection zones 2B′ and 2B″, the primary layer 2′ may further advantageously comprise a plurality of guiding means 2D′ which are substantially parallel with each other and define a secondary channel for guiding said liquid which connects between them said preferential collection zones 2B′ and 2B″ (FIG. 3).

Preferably substantially rectilinear and continuous, these guiding means 2D′ are, following the example of said guiding means 2C′, distinct from each other, that is to say that they are arranged at a distance from each other and do not touch each other. Advantageously, each of said guiding means 2D′ extends along a mean direction of longitudinal extension substantially parallel to the mean plane of extension of the primary layer 2′. Further advantageously, these guiding means 2D′ are substantially impermeable to said liquid, and preferably made of a material identical to that of said guiding means 2C′. Moreover, said guiding means 2D′ preferentially penetrate into the entire thickness of the primary layer 2′, and are of a width substantially similar (if not identical) to that of said guiding means 2C′.

In a particularly interesting manner, and in order to further improve the simultaneousness and homogeneity of the phenomenon of guiding said liquid to each of the preferential collection zones 2B′ and 2B″, said guiding means 2D′ advantageously pass right through said preferential collection zones 2B′ and 2B″, each guiding means 2D′ connecting one of the guiding means 2C′ adjacent to the preferential collection zone 2B′ to one of the guiding means 2C′ adjacent to the preferential collection zone 2B″. In other words, and as illustrated in FIG. 3, the barrier guiding means 2D′ are preferentially laid out parallel to each other to form a continuous secondary channel which passes through each of the preferential collection zones 2B′ and 2B″, while connecting two guiding means 2C′. Advantageously in this way, as is clear from FIG. 3, a secondary channel which on each of its ends is extended by a primary channel is obtained.

Said preferential collection zones 2B′ and 2B″ being distinct and preferentially positioned at a distance from each other along the mean plane of extension of the primary layer 2′, said primary layer 2′ preferably comprises a surface region (or an extent) of the primary layer 2′ which extends between said preferential collection zones 2B′ and 2B″. Separating the latter, said surface region advantageously connects the portions of the respective primary contours of said preferential collection zones 2B′ and 2B″.

In this respect, and to limit or even prohibit the displacement (or reflux) of said liquid from said surface region in the direction of at least one of the peripheral edges, said primary layer 2′ advantageously comprises a plurality of tertiary guiding means 2E′, each being respectively interposed between said surface region and at least one of the peripheral edges of the primary layer 2′. Said guiding means 2E′ are advantageously laid out to oppose the displacement of said liquid, from said surface region to at least one of the peripheral edges, via the part of the contour of said region not bordered by the preferential collection zones 2B′ and 2B″.

Preferably, in order to simplify as much as possible the design and the manufacture of said primary layer 2′, these guiding means 2E′ are respectively formed of the reunion of two guiding means 2C′, which are preferentially identical and symmetrical along the plane B-B′.

More generally, other conformations and configurations of said guiding means 2C, 2C′ (and, optionally 2D′ and/or 2E′) could be implemented without however going beyond the scope of the invention, from the moment that two adjacent means do not define between them a guiding channel for the liquid that would be convergent in the direction of at least one of the peripheral edges of the primary layer 2, 2′.

Preferentially, said primary piece 2A, 2A′ of textile, in one piece, is for its part preferentially 80% constituted of wood pulp (cellulose) and 20% of polyester fibres (for example PET), and has preferentially a grammage substantially equal to 28 g·m⁻². Such a primary piece 2A, 2A′ of textile may, for example, be obtained by wet or paper making technique (“wet laid” method), by incorporation of polyester fibres in a cellulose paste of wood pulp.

Secondary Layer

As illustrated in the figures, in an advantageous embodiment, the medical device 1, 1′ of the invention also comprises at least one secondary layer 3, 3′, distinct from said primary layer 2, 2′, integrating said biosensor 3E, 3E′, 3E″, the secondary layer being arranged with respect to the primary layer so that said biosensor extends facing the preferential collection zone.

Preferably laid out facing (advantageously directly in line with) said primary layer 2, 2′, said secondary layer 3, 3′ preferentially extends along a secondary plane of extension substantially parallel to the primary plane of extension of said primary layer 2, 2′. This secondary layer 3, 3′ advantageously corresponds to said second region of the aforementioned medical device 1, 1′, to which said liquid, coming from said primary layer 2, 2′, is directed and conveyed. If reference is made to the figures, the plumb line is advantageously given by the direction of the axis A-A′, B-B′, substantially orthogonal to the respective extension planes of the different layers of said medical device 1, 1′. In the preferential embodiments illustrated in said figures, said secondary layer 3, 3′ is advantageously laid out facing, directly in line with and above said primary layer 2, 2′. Preferably, said secondary layer 3, 3′ is absorbent vis-a-vis said liquid, that is to say that its component material(s) are advantageously provided with a non-zero absorption capacity of said liquid, and in an even more advantageous manner, a significant retention capacity of said liquid. According to the invention, this secondary layer 3, 3′ does not form a perfectly and strictly homogeneous layer at all points. Indeed, said secondary layer 3, 3′ itself comprises at least first 3A, 3A′ and second zones 3B, 3B′, which first 3A, 3A′ and second 3B, 3B′ zones are distinct from each other and each have different respective behaviours in contact with said liquid.

In a particularly interesting manner, these first 3A, 3A′ and second 3B, 3B′ zones are thus provided and designed to react differently, from a physical and/or chemical viewpoint, in the presence of the same said liquid with which they are placed in contact. From then on, said first 3A, 3A′ and second 3B, 3B′ zones advantageously constitute different functional zones with regard to the guiding of said liquid within the medical device 1, 1′. In particular, said first zone 3A, 3A′ preferentially corresponds to a zone of the secondary layer 3, 3′ to which said liquid, coming from said primary layer 2, 2′, will be primarily displaced, directed, whereas said second zone 3B, 3B′ advantageously preferably corresponds to another zone of the secondary layer 3, 3′ to which said liquid will also be directed, but in a lower priority manner however. In this sense, said first zone 3A, 3A′ advantageously constitutes a first “target” zone of interest for said liquid.

In this respect, said first zone 3A, 3A′ advantageously has a first rate of absorption and a first absorption capacity of said liquid, while said second zone 3B, 3B′ has a second rate of absorption and a second absorption capacity, said first absorption rate being strictly greater than said second absorption rate. Said first absorption capacity is for its part preferably strictly less than said second absorption capacity. “Absorption capacity” is here preferentially taken to mean the maximum volume of liquid that the material forming the considered zone 3A, 3B, 3A′, 3B′ of said secondary layer 3, 3′ can absorb therewithin, and advantageously retain. The term “absorption rate” preferentially refers for its part to the rapidity at which the material forming the considered zone 3A, 3B, 3A′, 3B′ is capable of absorbing a given quantity of said liquid. Such a differentiation in the rate and absorption capacity between said first 3A, 3A′ and second 3B, 3B′ zones may, for example, be conferred to the secondary layer 3, 3′ by localised modification (for example by compression) of the density of the material forming said secondary layer 3, 3′ or by the use, within the same said secondary layer 3, 3′, of materials of different nature and properties. These materials will be chosen in a suitable manner with regard to the desired absorption properties. In this way, it is thus possible to generate a competitive phenomenon between said first 3A, 3A′ and second 3B, 3B′ zones with regard to said liquid when the latter enter into contact with the secondary layer 3, 3′. Indeed, said first zone 3A, 3A′ “will attract” primarily said liquid to it, due to its greater absorption rate. In the first instance, the quasi-totality (or at least the majority by volume) of the liquid reaching the secondary layer 3, 3′ will thus be advantageously directed to the first zone 3A, 3A′ and absorbed by the latter. Then, once the first absorption capacity of said first zone 3A, 3A′ has been reached, that is to say once the latter is saturated with liquid (which may for example correspond to a volume of liquid substantially close to 1 ml), the liquid continuing to flow to the secondary layer 3, 3′ will then be “attracted” mainly (by volume) to said second zone 3B, 3B′, on account this time of the greater absorption capacity of the latter. Obviously, said first 3A, 3A′ and second zones 3B, 3B′ could have respective behaviours which differ from those described above, without however going beyond the scope of the invention.

Advantageously, the second zone 3B, 3B′ of the secondary layer 3, 3′ could be impregnated with a hydrophobic substance, such as for example EVA. This makes it possible to further lower said second absorption rate of the second zone 3B, 3B′ and favours the accumulation of said liquid in said first 3A, 3A′ zones rather than in said second 3B, 3B′ zones.

In an even more preferential manner, to facilitate and accelerate the displacement of said liquid from the primary layer 2, 2′ to said first 3A, 3A′ and second 3B, 3B′ zones of the secondary layer 3, 3′, said primary layer 2, 2′ advantageously has a third absorption capacity of said liquid, which is strictly less than, and preferably much less than, said first and second absorption capacities of the first 3A, 3A′ and second 3B, 3B′ zones of the secondary layer 3, 3′. Further preferably, said primary layer 2, 2′ advantageously has a third rate of absorption of said liquid, which is strictly greater, and preferably much greater, than said first and second rates of absorption of the first 3A, 3A′ and second 3B, 3B′ zones of the secondary layer 3, 3′.

Said first zone 3A, 3A′ of the secondary layer 3, 3′ comprises a biosensor 3E, 3E′, such as described previously, comprising a composition of agglomerated powders, such as described previously, comprising particles of EVA having a surface in part covered by at least one orthophosphoric acid salt and a visual indicator of microbiological growth.

According to the first preferential embodiment illustrated in FIGS. 2 and 6, the secondary layer 3 of the medical device 1 comprises a first zone 3A and a second zone 3B pursuant to the above. Preferably, as illustrated in the figures, said first zone 3A is delimited by a closed outer contour and is surrounded in a contiguous manner by said second zone 3B. In other words, said second zone 3B immediately adjoins said first zone 3A, bordering the latter over its whole periphery. However, said secondary layer could comprise more zones of different behaviours, without however going beyond the scope of the invention. Thus, according to the second preferential embodiment, illustrated in FIG. 3, the secondary layer 3′ of the medical device advantageously comprises a first zone 3A′ and a second zone 3B′, in accordance with the preceding, as well as a third zone 3A″. This third zone 3A″, advantageously distinct from said first zone 3A′ and second zone 3B′, may also for its part be delimited by a closed outer contour, and it is preferably surrounded in a contiguous manner by said second zone 3B′. Said third zone 3A″ has, preferably, properties and behaviour identical to those of said first zone 3A′, in particular in terms of absorption rate and capacity. However, it may fully be envisaged, without however going beyond the scope of the invention, that said first 3A′, second 3B′ and third 3A″ zones of the secondary layer 3′ each has a different respective behaviour in contact with said liquid. Said third zone 3A″ thus advantageously constitutes a second “target” zone of said secondary layer 3′. Said third zone 3A″ of the secondary layer 3′ comprises a biosensor 3E″, such as described previously, comprising a composition of agglomerated powders, such as described previously comprising particles of EVA having a surface in part covered with at least one orthophosphoric acid salt and a visual indicator of microbiological growth. The zones 3A′, 3A″ could comprise the same biosensor, or biosensors corresponding to different alternatives of the invention, notably comprising compositions of different agglomerated powders being able to comprise different visual indicators of microbiological growth.

Preferably, the thickness of the secondary layer 3, 3′ is substantially comprised between 2 and 5 mm. Advantageously, as illustrated in FIGS. 2, 3 and 6, said secondary layer 3, 3′ is not formed in one piece, that is to say of a single and same surface piece, but consists on the contrary of a surface assembly of distinct surface pieces. As illustrated in FIGS. 2 and 6, the secondary layer 3 is thus preferentially formed at least:
of a secondary piece 3C of textile provided with at least one first through window 3D arranged through said secondary piece 3C, said secondary piece 3C forming (or contributing at least to forming) said second zone 3B of the secondary layer 3, and
of a first tertiary piece 3E of textile, distinct from said secondary piece 3C and of shape substantially complementary to said first window 3D, said first tertiary piece 3E being closely housed (that is to say preferably “edge to edge”) within said first window 3D and forming (or contributing at least to forming) said first zone 3A of the secondary layer. This first tertiary piece 3E of textile is the biosensor described previously or comprises the biosensor described previously.

Alternatively, in the second preferential embodiment illustrated in FIG. 3, the secondary layer 3′ could advantageously be formed at least:
of a secondary piece 3C′ of textile provided with first 3D′ and second 3D″ through windows arranged through said secondary piece 3C′ and advantageously distinct and distant from each other,
and of first 3E′ and second 3E″ tertiary pieces of textile, distinct from each other and from said secondary piece 3C′, and respectively of shape substantially complementary to said first 3D′ and second 3D″ windows, these first 3E′ and second 3E″ tertiary pieces being respectively closely housed within said first 3D′ and second 3D″ windows 3D and forming (or contributing at least to forming) respectively said first 3A and third 3A″ zones of the secondary layer 3′.

Said first 3D′ and second 3D″ windows, on the one hand, and said first 3E′ and second 3E″ tertiary pieces, on the other hand, are preferably identical, but could quite obviously be different, without however going beyond the scope of the invention.

Said first 3E′ and second 3E″ tertiary pieces are a biosensor such as described previously or comprise a biosensor such as described previously.

Being advantageously in the form of surface blankets and preferentially chosen substantially of same thickness, said secondary 3C, 3C′ and tertiary 3E, 3E′, 3E″ pieces of textile are thus laid out in a coplanar manner to form (or at least contribute to forming) said secondary layer 3, 3′. Said first window 3D (respectively said first 3D′ and second 3D″ windows) and said corresponding first tertiary piece 3E (respectively said first 3E′ and second 3E″ tertiary pieces) may take respectively any known complementary shapes and, for example, take respectively a rectangular shape (as illustrated in FIGS. 2, 3 and 6) or yet again circular. Said secondary layer 3, 3′ and its first 3A, 3A′ and second 3B, 3B′ zones (or even third zone 3A″) are thus relatively simple to design and to manufacture, the choice of the materials forming respectively said first 3A, 3A′ and second 3B, 3B′ (or even third zone 3A″) zones being from that moment very vast, since said secondary layer 3, 3′ is not formed in one piece.

Preferably, said secondary piece 3C, 3C′ of textile is a piece of textile preferentially non-woven and hydrophilic and, in an even more preferential manner, needle punched. Needle punching of the textile indeed advantageously makes it possible to “aerate” the textile and, to a certain extent, to increase the absorption capacity of said secondary piece 3C, 3C′ (and thus to increase said second absorption capacity of said second zone 3B, 3B′). In an even more preferential manner, said secondary piece 3C, 3C′ of textile is 70% constituted of polyethylene fibres and 30% of polypropylene fibres, and has preferentially a grammage substantially equal to 220 g·m⁻².

Said first tertiary piece 3E (respectively said first 3E′ and second 3E″ tertiary pieces), is for its part preferentially a piece made of absorbent, hydrophilic material fixing, on the surface and/or within the thickness thereof, a composition of agglomerated powders comprising particles of ethylene vinyl acetate having a surface in part covered with at least one orthophosphoric acid salt and a visual indicator of microbiological growth. Said first tertiary piece 3E (respectively said first 3E′ and second 3E″ tertiary pieces), is advantageously the biosensor described previously. The biosensors forming the pieces 3E, 3E′ and 3E″ could be identical or different, according to the different embodiments described previously for the biosensor. In particular, the visual indicator of microbiological growth could be different or identical from one biosensor to another.

The composition of agglomerated powders of the biosensor described previously will advantageously be incorporated in the tertiary piece 3E (respectively said first 3E′ and second 3E″ tertiary pieces) on the surface, and at least in part within the thickness, of said first zone 3A (respectively said first 3A′ and third 3A″ zones). More precisely, said first zone 3A, 3A′ (or even third zone 3A″) preferably having a “lower” surface, intended to face said intermediate layer 4, 4′ and an opposite “upper” surface, intended to face said barrier layer 5, 5′ (or said adhesive sub-layer 6, 6′), the composition of agglomerated powders of the biosensor 3E, 3E′ (or even biosensor 3E″) will advantageously be laid out in the vicinity of said “upper” surface and at least in part within the thickness of said first zone 3A, 3A′ (or even third zone 3A″). In an even more preferential manner, said first zone 3A, 3A′ (or even third zone 3A″) being delimited by a closed outer contour, said composition of agglomerated powders of the biosensor 3E, 3E′ (or even biosensor 3E″) will advantageously be positioned at a distance from said outer contour, preferably substantially at the centre of said first zone 3A, 3A′ (or even third zone 3A″), defining a zone 3F, 3F′ (or even 3F″) of the biosensor 3E, 3E′ (or even biosensor 3E″) comprising said composition of agglomerated powders. Thus, when said liquid reaches said first zone 3A, 3A′ (or even third zone 3A″), the composition of agglomerated powders of the biosensor 3E, 3E′ (or even biosensor 3E″) is then advantageously surrounded by said liquid impregnating the first zone 3A, 3A′ (or even third zone 3A″), such that the composition of agglomerated powders of the biosensor comes into contact with said liquid in a substantially homogeneous manner along its entire periphery.

Intermediate Layer

As illustrated in the figures, the medical device 1, 1′ of the invention further advantageously comprises an intermediate layer 4, 4′, which is interposed between said primary 2, 2′ and secondary 3, 3′ layers. Laid out facing, directly in line with, said primary 2, 2′ and secondary 3, 3′ layers respectively, said intermediate layer 4, 4′ preferably extends along an intermediate plane of extension substantially parallel to the primary and secondary planes of extension of said primary 2, 2′ and secondary 3, 3′ layers. In the examples illustrated in the figures, said intermediate layer 4, 4′ is advantageously laid out on the one hand facing, directly in line with and above (and advantageously in direct surface contact with) said primary layer 2, 2′ and, on the other hand, facing, directly in line with and below (and advantageously in direct surface contact with) said secondary layer 3, 3′.

According to the invention, the intermediate layer 4, 4′ itself comprises at least one first portion impermeable 4A, 4A′ to said liquid (that is to say leak tight to the latter) and at least one first portion 4B, 4B′ permeable to said liquid, the latter enabling the passage, therethrough, of said liquid coming from the primary layer 2, 2′ to the secondary layer 3, 3′, whereas said first impermeable portion 4A, 4A′ prohibits on the contrary the passage of the liquid therethrough. Preferably, said first impermeable portion 4A, 4A′ and said first permeable portion 4B, 4B′ are respectively continuous.

In the first preferential embodiment of FIGS. 1, 2, 5 and 6, said intermediate layer 4 preferably comprises, apart from said first impermeable portion 4A and said first permeable portion 4B, a second impermeable portion 4C, advantageously distinct from the first impermeable portion 4A. Alternatively, in the second preferential embodiment of FIG. 3, said intermediate layer 4′ preferably comprises, apart from said first impermeable portion 4A′ and said first permeable portion 4B′, second 4C′ and third 4A″ impermeable portions, as well as a second permeable portion 4B″. Preferably, these second 4C′ and third 4A″ impermeable portions are each distinct from the first impermeable portion 4A′, and the second permeable portion 4B″ is advantageously distinct from the first permeable portion 4B′.

Such permeability/impermeability characteristics with regard to a liquid of given nature and composition may be obtained by any known technique, for example by locally modulating the porosity or the solubility in said liquid of the material forming said intermediate layer 4, 4′. However, said first permeable portion 4B (alternatively, said first 4B′ and second 4B″ permeable portions) is preferentially formed by at least one first intermediate through opening 4D (respectively, by at least one first 4D′ and at least one second 4D″ intermediate through openings) made through said intermediate layer 4, 4′. Said first intermediate opening 4D (respectively said first 4D′ and second 4D″ intermediate openings) will obviously be dimensioned in a suitable manner to allow effectively said liquid to pass therethrough. Said first impermeable portion 4A, 4A′ of the intermediate layer 4, 4′ (and also preferably the second impermeable portion 4C, 4C′, or even also advantageously the third impermeable portion 4A″) is for its part preferentially formed of a preferentially continuous film (or layer), made of hydrophobic polymer material, for example of pressure sensitive adhesive (PSA) (for example silicone based) or polyurethane type. Said intermediate layer 4, 4′ is thus of very simple design and could advantageously be produced easily, as will be detailed hereafter, by partial coating of a face of one or the other of said primary 2, 2′ and secondary 3, 3′ layers using said pressure sensitive adhesive or polyurethane. It will furthermore be noted that preferentially resorting to a pressure sensitive adhesive will advantageously make it possible to make integral with each other, thanks to said intercalary intermediate layer 4, 4′ thus formed, said primary 2, 2′ and secondary 3, 3′ layers, once the latter advantageously laid out one facing the other pursuant with the preceding.

In addition, in the preferential case, illustrated in the figures, where the first zone 3A (alternatively the first zone 3A′, as well as preferably the third zone 3A″) is delimited by a closed outer contour and is surrounded in a contiguous manner by said second zone 3B, 3B′, the first intermediate opening 4D (alternatively the first intermediate opening 4D′, as well as preferably the second intermediate opening 4D″) advantageously forms a curvilinear first port 4D (respectively a first port 4D′, as well as preferably the second port 4D″) closed on itself. In other words, the first intermediate opening 4D (alternatively the first intermediate opening 4D′, as well as preferably the second intermediate opening 4D″) is, in this case, in the form of a first port 4D (respectively in the form of a first port 4D′, as well as preferably a second port 4D″) or a “channel” of predefined width, which extends in the intermediate plane of extension of the intermediate layer 4, 4′. Closed on itself at its ends, this “channel” emerges in a continuous manner over the whole length of its closed profile, on either side of the surface of the intermediate layer 4, 4′ .

In a particularly advantageous manner, the closed curvilinear profile of this first intermediate opening 4D, 4D′ (or also and advantageously even the second opening 4D″) is conjugated with the respective profile of the closed outer secondary contour of the first zone 3A, 3A′ (or even the third zone 3A″) of the secondary layer 3, 3′. Thus, if said outer secondary contour of the first zone 3A, 3A′ is for example circular or rectangular, said corresponding first intermediate opening 4D, 4D′ then forms a first port 4D, 4D′, annular or annular with inner and outer rectangular contours (FIGS. 2, 3 and 6). Thus, in the first preferential embodiment of FIGS. 2 and 6, the first intermediate opening 4D of the intermediate layer 4 is preferably in the form of an annular port 4D with inner and outer rectangular contours, of profile matched with the rectangular shape taken by the first zone 3A. In the second preferential embodiment of FIG. 3, said first 4B′ and a second 4B″ permeable portions are respectively formed of first 4D′ and second 4D″ intermediate openings. These first 4D′ and second 4D″ intermediate openings, on the one hand, and said first 3A′ and third 3A″ zones, on the other hand, are preferentially identical to each other, and said first 4D′ and second 4D″ intermediate openings are preferably in the form of first 4D′ and second 4D″ annular ports with inner and outer rectangular contours, of respective profiles matched with the rectangular shape respectively preferentially taken by said first 3A and third zones 3A″. The average width of said first 4D, 4D′ and second 4D″ ports, that is to say the distance separating the inner contour and the outer contour of the latter, is for example comprised between 3 and 5 mm.

Preferably, the thickness of the intermediate layer 4, 4′ is chosen less than, or even much less than, the respective thicknesses of the primary 2, 2′ and secondary 3, 3′ layers, so as to guarantee good compactness of said medical device 1, 1′. Typically, the thickness of the intermediate layer 4, 4′ thus could be substantially comprised between 0.05 and 0.3 mm. Depending on the thickness chosen for the intermediate layer 4, 4′ and the very nature of said considered liquid, said first intermediate opening 4D, 4D′ (or even also advantageously the second opening 4D″) could optionally be filled by a complementary element permeable to said liquid (for example a surface piece of non-woven hydrophilic textile), so as to facilitate the passage of said liquid through the first permeable portion 4B, 4B′ (or even also advantageously through the second permeable portion 4B″) thus obtained by forming a “bridge” between said primary 2, 2′ and secondary 3, 3′ layers. Alternatively, said intermediate opening(s) 4D, 4D′, 4D″ could be left empty, without prejudice for the correct passage of said liquid, in so far as said primary 2, 2′ and secondary 3, 3′ layers between which said intermediate layer 4, 4′ is interposed could come locally into contact or quasi contact with each other at the level of said first intermediate opening 4D, 4D′ (or even also advantageously the second opening 4C″) under the effect of their respective weights.

Said primary layer 2, 2′, secondary layer 3, 3′ and intermediate layer 4, 4′ of the medical device 1, 1′ of the invention thus being described in detail, the respective configuration and the relative layout of these layers 2, 3, 4, 2′, 3′, 4′ with respect to each other within said medical device 1, 1′ will now be described more precisely. Their respective functions and contributions to the operation of the medical device 1, 1′ of the invention will thus be understood.

According to the invention, and as illustrated in the figures, said first impermeable portion 4A, 4A′ extends facing (that is to say in front of), and preferably directly in line with, said first zone 3A, 3A′ of the secondary layer 3, 3′, the projection Pr1 (orthogonal) of the first impermeable portion 4A, 4A′ in a plane P (fictive) of projection parallel to said secondary 3, 3′ and intermediate 4, 4′ layers covering at least half of the surface of the projection Pr2 (orthogonal) of the first zone 3A, 3A′ in this same plane of projection, to limit reflux of said liquid from the first zone 3A, 3A′ of the secondary layer 3,3′ to the primary layer 2, 2′. In other words, as is clearly shown in FIG. 4, the surface area of the coverage zone Zr of the respective projections Pr1, Pr2 of the first impermeable portion 4A, 4A′ and first zone 3A, 3A′ in the plane P is at least equal to half the surface area of the projection Pr2 of the first zone 3A, 3A′. This signifies that said secondary 3, 3′ and intermediate 4, 4′ layers are configured and laid out with respect to each other within the medical device 1, 1′ in such a way that said first zone 3A, 3A′ at least mainly faces said first impermeable portion 4A, 4A′. Thus, said first zone 3A, 3A′ and said first impermeable portion 4A, 4A′ being at least mainly placed in correspondence along a direction parallel to the axis A-A′, B-B′ orthogonal to the layers, said intermediate layer 4, 4′ advantageously makes at least half of the surface of the first zone 3A, 3A′ inaccessible to said liquid along a direction orthogonal to the secondary 3, 3′ and intermediate 4, 4′ layers. It is therefore readily understandable that the reflux, and in particular the “direct” reflux (that is to say along a path substantially linear and orthogonal to the primary 2, 2′, secondary 3, 3′ and intermediate 4, 4′ layers), of said liquid from said first zone 3A, 3A′ of the secondary layer 3, 3′ to said primary layer 2, 2′, via the intermediate layer 4, 4′, is thus particularly controlled and limited. Preferably, the projection Pr1 (orthogonal) of the first impermeable portion 4A, 4A′ in said plane P of projection covers at least 55%, further preferably at least 60%, and in an even more preferential manner at least 80% of the surface of the projection Pr2 (orthogonal) of the first zone 3A, 3A′ in this same plane P, in order to optimise the desired anti-reflux effect. According to a particularly advantageous alternative, visible in FIG. 6, the projection Pr1 of the first impermeable portion 4A, 4A′ in said plane P parallel to said secondary 3, 3′ and intermediate 4, 4′ layers is merged with the projection Pr2 of the first zone 3A, 3A′ in this same plane P (100% coverage), Thus, the “direct” reflux of liquid from the first zone 3A, 3A′ of the secondary layer 3, 3′ to the primary layer 2, 2′, via the intermediate layer 4, 4′, is advantageously prohibited.

In the preferential embodiment of FIG. 3, whereas said first impermeable portion 4A′ extends facing (and preferably directly in line with) said first zone 3A′, said third impermeable portion 4A″ preferentially extends facing, and preferably directly in line with, said third zone 3A″, the projection (orthogonal) of the third impermeable portion 4A″ in a plane (fictive) of projection parallel to said secondary 3′ and intermediate 4′ layers covering at least half of the respective surface of the projections (orthogonal) of the first zone 3A′ and the third zone 3A″ in this same plane of projection. Given the above, it will thus be understood that the reflux (and in particular the “direct” reflux) of liquid from each of said first 3A′ and third 3A″ zones of the secondary layer 3′ to said primary layer 2′, via the intermediate layer 4′, is thus particularly controlled and limited. Preferably, the projection (orthogonal) of the third impermeable portion 4A″ in said plane of projection covers at least 55%, further preferably at least 60%, and in an even more preferential manner 80% of the surface of the projection (orthogonal) of the third zone 3A″ in this same plane. According to an alternative (not illustrated), the projection of the third impermeable portion 4A″ in said plane of projection parallel to said secondary 3′ and intermediate 4′ layers is itself merged with the respective projection of the third zone 3A″ in this same plane (100% coverage).

Said intermediate layer 4, 4′ preferably comprises (as mentioned above), a second portion 4C, 4C′ impermeable to said liquid. Advantageously, this second impermeable portion 4C, 4C′ extends facing, that is to say in front of, said second zone 3B, 3B′ of the secondary layer 3, 3′, the projection (orthogonal) of the second impermeable portion 4C, 4C′ in a plane (fictive) of projection parallel to said secondary 3, 3′ and intermediate 4, 4′ layers covering at least half of the surface of the projection (orthogonal) of the second zone 3B, 3B′ in this same plane of projection, thus advantageously to limit the reflux (and in particular the “direct” reflux) of said liquid from said second zone 3B, 3B′ to said primary layer 2, 2′. In other words, said secondary 3, 3′ and intermediate 4, 4′ layers are advantageously configured and laid out with respect to each other within said medical device 1, 1′ in such a way that the second zone 3B, 3B′ at least mainly faces the second impermeable portion 4C, 4C′. Thus, the second zone 3B, 3B′ and the second impermeable portion 4C, 4C′ being at least mainly placed in correspondence along a direction parallel to the axis A-A′, B-B′ orthogonal to the layers, said intermediate layer 4, 4′ advantageously makes at least half of the surface of the second zone 3B, 3B′ inaccessible to said liquid along a direction orthogonal to the secondary 3, 3′ and intermediate 4, 4′ layers. Further preferably, the projection (orthogonal) of the second impermeable portion 4C, 4C′ in said plane of projection covers at least 55%, further preferably at least 60%, or even at least 80% of the surface of the projection (orthogonal) of the second zone 3B, 3B′ in this same plane, in order to procure a sufficient anti-reflux effect.

According to an alternative, visible in FIGS. 1 to 3, the projection (orthogonal) of said first permeable portion 4B, 4B′ in a plane (fictive) of projection parallel to said secondary 3, 3′ and intermediate 4, 4′ layers overlaps, that is to say covers simultaneously and at least partially, the respective projections (orthogonal) of the first 3A, 3A′ and second 3B, 3B′ zones in this same plane of projection. In other words, said secondary 3, 3′ and intermediate 4, 4′ layers are designed and laid out with respect to each other within said medical device 1, 1′ so that said first permeable portion 4B, 4B′ is placed in correspondence both with a part of the surface of the first zone 3A, 3A′ and with a part of the surface of the second zone 3B, 3B′. As is clear from the figures, said first permeable portion 4B, 4B′ thus advantageously extends both under and in direct contact with a part of the surface of the first zone 3A, 3A′ and with a part of the surface of the second zone 3B, 3B′.

In the preferential embodiment of FIG. 3, wherein said intermediate layer 4′ preferentially comprises (as mentioned above) preferably first 4B′ and second 4B″ permeable portions distinct from each other, the respective projection (orthogonal) of these first 4B′ and second 4B″ permeable portions in a plane (fictive) of projection parallel to the layers advantageously overlaps, that is to say covers respectively at least partially, the projections (orthogonal) of the first 3A′ and second 3B′ zones, and the projections (orthogonal) of the third 3A″ and second 3B′ zones in this same plane of projection. In other words, said first permeable portion 4B′ advantageously extends both facing, and preferably in direct contact with a part of the surface of the first zone 3A′ and with a part of the surface of the second zone 3B′.

Respectively, said second permeable portion 4B′ advantageously extends both facing, and preferably in direct contact with, a part of the surface of the third zone 3A″ and with a part of the surface of the second zone 3B′.

In an even more preferential manner, said first zone 3A, being advantageously delimited by a closed outer contour and being surrounded in a contiguous manner by said second zone 3B, and said first intermediate opening 4D advantageously forming a first curvilinear port 4D closed on itself, such as envisaged hereabove, the projection (orthogonal) of said contour of the first zone 3A in a plane (fictive) parallel to said secondary 2 and intermediate 4 layers is superimposed in a continuous manner on the projection (orthogonal) of said corresponding first curvilinear port 4D in this same parallel plane (FIG. 2). In other words, said secondary 3 and intermediate 4 layers are preferentially configured and laid out with respect to each other within said medical device 1 so that the orthogonal projection, in a plane parallel to said layers, at any point of the contour of the first zone 3A is contained in the orthogonal projection of the corresponding curvilinear port 4D in this same plane. In this way, said intermediate layer 4 advantageously allows, by means of said permeable portion 4B, the simultaneous passage of said liquid from the primary layer 2 to one or the other of said first 3A and second 3B zones of the secondary layer 3, whereas the reflux of the liquid from these first 3A and second 3B zones to the primary layer 2 is substantially limited by the presence of said impermeable portion 4A.

A configuration in all points similar will advantageously be retained as regards the second embodiment of FIG. 3. Thus, said first 3A′ and third 3A″ zones each being advantageously delimited by a closed outer contour and surrounded in a contiguous manner by said second zone 3B′, and said first 4D′ and second 4D″ intermediate openings respectively forming first 4D′ and second 4D″ curvilinear ports closed on themselves, the respective projection (orthogonal) of the contour of the first 3A′ and third 3A″ zones in a plane (fictive) parallel to said secondary 2, 2′ and intermediate 4, 4′ layers is superimposed in a continuous manner on the respective projection (orthogonal) of the corresponding first 4D′ and second 4D″ curvilinear ports in this same parallel plane. In this way, the intermediate layer 4′ advantageously allows, through respectively first 4B′ and second 4B″ permeable portions, the simultaneous passage of said liquid from the primary layer 2, 2′ to each of the first 3A′, second 3B′ and third 3B″ zones of the secondary layer 3′, whereas the reflux of said liquid from the latter to the primary layer 2′ is substantially limited by the presence of said impermeable portion 4A′.

FIGS. 5 and 6 illustrate another alternative, particularly advantageous. In this alternative, said projection Pr1 (orthogonal) of the first impermeable portion 4A in said plane P (fictive) parallel to said secondary 3 and intermediate 4 layers is merged with the projection Pr2 of the first zone 3A in this same plane, as has been envisaged above. Moreover, said first zone 3A is preferentially delimited by a closed outer contour and is surrounded in a contiguous manner by said second zone 3B, said first intermediate opening 4D forming a first curvilinear port 4D closed on itself. On the other hand, unlike the aforementioned alternative, and as illustrated in FIGS. 5 and 6, the projection of said first curvilinear port 4D in a plane parallel to said secondary 3 and intermediate 4 layers surrounds in a contiguous manner the respective projection of said contour of the first zone 3A in this same plane. In other words, said secondary 3 and intermediate 4 layers are preferentially designed and laid out with respect to each other within said complex 1 such that the orthogonal projection, in a plane parallel to said layers 3, 4, an any point of the outer contour of the first zone 3A is merged with the orthogonal projection of the inner contour of the corresponding curvilinear port 4D in this same plane.

By analogy, this particular configuration could advantageously be transposed to the embodiment of FIG. 3. Thus, said first 3A′ and third 3A″ zones being advantageously each delimited by a closed outer contour and surrounded in a contiguous manner by said second zone 3B′, and said first 4D′ and second 4D″ intermediate openings respectively forming first 4D′ and second 4D″ curvilinear ports closed on themselves, the respective projection (orthogonal) of said first 4D′ and second 4D″ curvilinear ports in a plane parallel to said secondary 3′ and intermediate 4′ layers will then surround in a contiguous manner the respective projection of the respective outer contours of the first 3A′ and third 3A″ zones in this same plane.

Thus, the first permeable portion 4B (alternatively the first permeable portion 4B′, as well as preferably the second permeable portion 4B″) will advantageously be laid out facing uniquely said second zone 3B (respectively said second zone 3B′).

A configuration according to such an alternative makes it possible to obtain a very good compromise between, on the one hand, a certain limitation of the progression of the liquid from the primary layer 2, 2′ to the first zone 3A, 3A′ (or even also to the third zone 3A″) of the secondary layer 3, 3′, on account of the absence of direct access to said first zone 3A, 3A′ and, on the other hand, an optimal control of the problem of reflux of said liquid from the first zone 3A, 3A′ (or even from the third zone 3A″) to the primary layer 2, 2′.

Preferably, as illustrated in FIGS. 2 and 6, said permeable portion 4B of the intermediate layer 4 is positioned substantially facing, and preferably directly in line with, the preferential collection zone 2B. Alternatively, as illustrated in FIG. 3, the first 4B′ and second 4B″ permeable portions of the intermediate layer 4′ are preferably respectively positioned substantially facing, and preferably directly in line with, the first 2B′ and the second 2B″ preferential collection zones. This signifies that the primary layer 2, 2′ and the intermediate layer 4, 4′ are, for their part, advantageously configured and laid out with respect to each other in such a way that the first permeable portion 4B (alternatively the first 4B′ and second 4B″ permeable portions) and the preferential collection zone 2B (respectively the first 2B′ and second 2B″ preferential collection zones) are placed in correspondence (respectively two by two) along a direction parallel to the axis A-A′, B-B′ orthogonal to the layers. Thus, only the part of said liquid impregnating the primary layer 2, 2′ which is found at the level of the preferential collection zone(s) 2B, 2B′, 2B″ of the primary layer 2, 2′ is capable of being collected and of passing through said intermediate layer 4, 4′ in the direction of said secondary layer 3, 3′.

Barrier Layer

According to the invention, the medical device 1, 1′ further advantageously comprises a barrier layer 5, 5′ which covers, preferably integrally, said secondary layer 3, 3′.

As illustrated in the figures, this barrier layer 5, 5′ is advantageously positioned facing, and preferably directly in line with, the secondary layer 3, 3′, such that it constitutes one of the most superficial layers of said medical device 1, 1′. Hence, the secondary layer 3, 3′ is thus advantageously interposed between said barrier layer 5, 5′ and the intermediate layer 4, 4′. This barrier layer 5, 5′ is substantially impermeable to said liquid, such that when said liquid has reached the secondary layer 3, 3′, coming from the primary layer 2, 2′ and via the intermediate layer 4, 4′, advantageously it cannot leak, flow, out of the medical device 1, 1′ to the medium external to said medical device 1, 1′ along a direction secant to the mean plane of extension of said barrier layer 5, 5′. Advantageously, said barrier layer 5, 5′ is also impermeable to liquid water (in so far as said liquid is not itself an aqueous liquid), in order to prevent any passage of water from the medium external to the medical device 1, 1′ to the inside of the latter through said barrier layer 5, 5′. Preferably, said barrier layer 5, 5′ is on the other hand permeable to air, and in particular to dioxygen and water vapour, such that it is advantageously “breathable”. Further preferably, said barrier layer 5, 5′ is transparent to visible light, that is to say that the material which forms the barrier layer 5, 5′ is substantially translucid, which advantageously makes it possible to observe, through said barrier layer 5, 5′, the aspect of the underlying secondary layer 3, 3′ and, in particular, the biosensor present in the first zone 3A, 3A′ or third zone 3A″ of the latter, through said barrier layer 5, 5′. Preferably, said barrier layer 5, 5′ is formed of a barrier film 5A, 5A′ made of flexible material, for example made of microporous polyurethane. Depending on the material employed, the average thickness of the barrier layer 5, 5′ could be substantially comprised between 20 and 50 μm.

The barrier layer 5, 5′ could be assembled, laid out, overlapping on the secondary layer 3, 3′ in direct contact with the latter or instead, optionally, using an adhesive sub-layer 6, 6′, continuous or not, and interposed between said secondary layer 3, 3′ and said barrier layer 5, 5′. This adhesive sub-layer 6, 6′ will be preferentially formed of a thin film 6A, 6A′ of an adhesive material (for example a pressure sensitive adhesive (PSA), for example rubber or silicone based). Advantageously, such a sub-layer 6, 6′ will then itself be chosen permeable to air (for example in the form of a discontinuous or microperforated thin film) and transparent, or even optionally impermeable to said liquid. In the preferential case, mentioned above, where said secondary layer 3, 3′ is not formed in one piece, but consists on the contrary of a surface assembly of distinct surface secondary 3C, 3C′ and tertiary 3E, 3E′, 3E″ pieces, said barrier layer 5, 5′ (and, preferably, said adhesive sub-layer 6 , 6′) will advantageously make it possible to maintain the relative layout of said secondary 3C, 3C′ and tertiary 3E, 3E′, 3E″ pieces.

Preferentially, the barrier layer 5, 5′ extends laterally so as to extend beyond the underlying layers and to define a peripheral zone corresponding to the part extending beyond the other layers. Said peripheral zone is, when the medical device 1, 1′ is positioned on the skin, the mucous membranes, directly facing the skin, the mucous membranes. In a preferred manner, the peripheral zone extends over at least two opposite sides of the medical device 1, 1′, in other words extends over either side of the other layers. This peripheral zone is on the lower face (on the side of the other layers) of the barrier layer advantageously coated, in a continuous or discontinuous manner, with an adhesive enabling the adhesion of the medical device 1, 1′ on the skin, the mucous membranes and its maintaining in contact with the wound.

The adhesive may notably be a pressure sensitive adhesive (PSA) or a silicone based gel.

The term “pressure sensitive adhesive” (PSA), such as it is used in the present paper relates to adhesives which can adhere to a surface and be unstuck therefrom without there being transfer of notable quantities of adhesive onto the surface and which may be again stuck on the same or another surface because the adhesive conserves a part or the totality of its tack and its adhesion force.

Among adhesives widely used in medical devices and which are in contact with the skin, it is possible to cite pressure sensitive adhesives based on silicone which are capable of adhering to a surface simply by contact or under the effect of a light pressure. They have considerable advantages compared to acrylic adhesives. Indeed, acrylic adhesives may not only cause irritation of the skin in certain patients, but they also have a tendency to increase the adherence of the skin over time, making the repositioning of the medical device awkward. Silicone PSAs are ideally suited to the increasing needs of new medical devices due to their biocompatibility and their permeability enabling the diffusion of oxygen, carbon dioxide and water vapour, which makes them perfectly suited to medical applications in which increased aeration is necessary. However, the adhesion to the skin must be maintained in a zone of comfort for the patient in order to avoid the sensory discomfort linked to the phases of detachment of the medical device.

For medical devices, from the moment that the aspect contact with the skin is required, the sterility of the material in contact with the skin is a major safety criterion for a large number of medical devices and cannot be omitted because its absence may be heavy with consequences.

Sterilisation by ionisation is a technique that is particularly intended for thermosensitive materials. Sterilisation by gamma (y) rays is widely used in the medical field. A radiation (doses comprised between 15 and 50 kGy) is emitted by a synthetic cobalt 60 which leads to the destruction of the DNA and the RNA of pathogenic agents, preventing their replication and gene expression. This method has a major drawback linked to the fact that certain materials may be damaged by this gamma radiation. Indeed, concerning silicone gels, the ionising radiation used for the sterilisation have a particularly detrimental effect notably on their properties of adhesion to the skin (or “tack”).

In an advantageous alternative of the invention, the adhesive described in the application FR 17/00725 will be used (not yet published, filed on the 7 Jul. 2017). This adhesive is a silicone adhesive, which after sterilisation by gamma (γ) ray, maintains its properties of adhesion suited to use on skin.

This pressure sensitive silicone adhesive Z is obtained by cross-linking a silicone composition X comprising:

1) from 80 to 20 parts by weight of at least one silicone resin A comprising SiOH functions,

2) from 20 to 80 parts by weight of at least one polyorganosiloxane G1 comprising at least two end of chain SiOH functions or at least one polyorganosiloxane G2 comprising at least two end of chain Si-vinyl functions and having a viscosity comprised between 20,000 and 600,000 mPa·s when it is a silicone oil or having a consistency at 25° C. comprised between 200 mm/10 and 2000 mm/10 when it is a silicone gum, and

3) at least one solvent S,

4) a silicone base B1 capable of reacting by addition reactions when polyorganosiloxane G2 is present, and

5) optionally a condensation catalyst C1 when polyorganosiloxane G1 is present,

with the condition according to which the quantity of solvent S is determined in such a way that the silicone composition X contains a solid content of silicone by weight from 20 to 80%, and preferably from 40 to 70%.

The viscosities of which it is question in the present description correspond to a magnitude of dynamic viscosity at 25° C. referred to as “Newtonian”, that is to say the dynamic viscosity which is measured, in a manner known per se, with a Brookfield viscometer with a sufficiently low shear rate gradient so that the measured viscosity is independent of the rate gradient (measurement carried out according to the ASTM D445-06 standard, 2006). The term gum is used for organosilicon compounds having viscosities conventionally greater than ˜600,000 mPa·s which corresponds to a molecular weight above 260,000 g·mol⁻¹. The consistency or penetrability of a gum is determined at 25° C. using a PNR12 type penetrometer or equivalent model making it possible to apply a cylindrical head on the sample under normalised conditions. The penetrability of a gum is the depth expressed in tenths of millimetres at which a calibrated cylinder penetrates into the sample for one minute. To this end, a sample of gum is introduced into an aluminium cup of 40 mm diameter and 60 mm height. The cylindrical head made of bronze or brass measures 6.35 mm diameter and 4.76 mm height and is borne by a metal rod 51 mm long and 3 mm diameter which is adapted to the penetrometer. This rod is weighted by an additional load of 100 g. The total weight of the assembly is 151.8g of which 4.3g for the cylindrical piece and its rod support. The cup containing the sample of gum is placed in a thermostatic bath at 25±0.5 ° C. for at least 30 min. The measurement is carried out by following the manufacturer's instructions. The values of the depth (V) in tenths of millimetres and the time (t) in seconds to reach this depth are indicated on the apparatus. The penetrability is equal to 60V·t⁻¹ expressed in tenths of millimetres per minute.

In a first embodiment, the silicone composition X comprises:

1) from 80 to 20 parts by weight of at least one silicone resin A comprising SiOH functions,

2) from 20 to 80 parts by weight of at least one polyorganosiloxane G1 comprising at least two end of chain SiOH functions which is a silicone gum having a consistency at 25° C. comprised between 200 mm/10 and 2000 mm/10,

3) optionally a condensation catalyst C1, and

4) at least one solvent S,

with the conditions according to which:

a) the silicone gum G1/silicone resin A ratio by weight is comprised between 0.60 and 1.00, and preferably comprised between 0.70 and 0.90, and

b) the quantity of solvent is determined in such a way that the silicone composition X contains a solid content by weight of silicone of 20 to 80%, and preferably of 40 to 70%.

In a second embodiment, the silicone composition X comprises:

1) from 80 to 20 parts by weight of at least one silicone resin A comprising SiOH functions,

2) from 20 to 80 parts by weight of at least one polyorganosiloxane G2 comprising at least two end of chain Si-vinyl functions and which is a silicone gum having a consistency at 25° C. comprised between 200 mm/10 and 2000 mm/10,

3) a silicone base B1 capable of reacting by addition reactions comprising:
at least one organohydrogen siloxane having at least 2 SiH functions in a sufficient quantity to provide a SiH/Sivinyl molar ratio comprised between 0.5:1 and 20:1,
an addition reaction catalyst C2, and
optionally an addition reaction inhibitor, and

4) at least one solvent S,

with the condition according to which the quantity of solvent S is determined in such a way that the silicone composition X contains a solid content by weight of silicone of 20 to 80%, and preferably of 40 to 70%.

In one or the other of these embodiments, the silicone resin A comprising SiOH functions is advantageously chosen from the group constituted by:

a) hydroxylated silicone resins of MQ^{(OH)} type which are copolymers comprising siloxy motifs M and Q^{(OH)} of following formulas:

M=R¹R²R³SiO_{1/2}, and

Q^{(OH)}=(OH)SiO_{3/2},
with optionally the presence of a siloxy motif Q=SiO_{4/2}

b) hydroxylated silicone resins of MD^{Vi}Q^{(OH)} type which are copolymers comprising siloxy motifs M, D^{Vi} and Q^{(OH)} of following formulas:

M=R¹R²R³SiO_{1/2},

D^{Vi}=(Vi)(R¹)SiO_{2/2}, and

Q^{(OH)}=(OH)SiO_{3/2},
with optionally the presence of a siloxy motif Q=SiO_{4/2}

c) hydroxylated silicone resins of MM^{Vi}Q^{(OH)} type which are copolymers comprising siloxy motifs M, M^{Vi} and Q^{(OH)} of following formulas:

M=R¹R²R3SiO_{1/2},

M^{Vi}=(Vi)(R¹)(R²)SiO_{2/2}, and

Q^{(OH)}=(OH)SiO_{3/2},
with optionally the presence of a siloxy motif Q=SiO_{4/2}

d) hydroxylated silicone resins of MDT^{(OH)} type which are copolymers comprising siloxy motifs M, D and T^{(OH)} of following formulas:

M=R¹R²R³SiO_{1/2},

D=R¹R²SiO_{2/2},

T^{(OH)}=(OH)R¹SiO_{2/2}, and

e) hydroxylated silicone resins of DT^{(OH)} type which are copolymers comprising siloxy motifs D and T^{(OH)} of following formulas:

D=R¹R²SiO_{2/2},

T^{(OH)}=(OH)R¹SiO_{2/2},

formulas in which the symbol Vi=a vinyl group, the symbols R¹, R² and R³ are chosen independently of each other among:
linear or branched alkyl groups having 1 to 8 carbon atoms included and optionally substituted by one or more halogen atoms, and preferably chosen from groups constituted of methyl, ethyl, isopropyl, tertiobutyl and n-hexyl groups, and
aryl or alkylaryl groups having 6 to 14 carbon atoms included, and preferably chosen from the groups constituted of phenyl, xylyl and tolyl groups.

The adhesive described in the application FR 17/00725 and described previously may also be used in the different layers of the medical device 1,1′ each time that mention is made of a pressure sensitive adhesive (PSA).

Optional Other Layers

Preferably, and as illustrated in the figures, the medical device 1, 1′ further comprises a transfer layer 7, 7′ permeable to said liquid. Arranged parallel facing, and preferably directly in line with, said primary layer 2, 2′, and in a manner opposite to said intermediate layer 4, 4′ with respect to said primary layer 2, 2′, this transfer layer 7, 7′ enables the passage of said liquid coming from the medium external to said medical device to said primary layer 2, 2′. Said transfer layer 7, 7′ preferably constitutes a superficial layer of said medical device 1, 1′, opposite to said barrier layer 5, 5′. Preferably, said transfer layer 7, 7′ is formed of a surface meshed blanket 7A, 7A′, preferably made of polymer material, for example silicone based (for example of liquid silicone resin (LSR) type), of which the average size of the meshes confers on said transfer layer 7, 7′ its character of permeability to said liquid. Preferably, the thickness of the transfer layer 7, 7′ is substantially comprised between 0.01 and 0.5 mm.

The transfer layer 7, 7′ of said medical device 1, 1′ could preferentially be formed of a meshed blanket 7A, 7A′ made of silicone based polymer material having a low degree of tack, so that said transfer layer 7, 7′ does not adhere substantially to the wound (and to the immediately surrounding skin and/or mucous membranes) against which said medical device will be positioned. The polymer material in question will be advantageously chosen compatible from a sanitary, and in particular dermatological, viewpoint. Thus, such a medical device is particularly comfortable and safe to use, the removal of said medical device being substantially atraumatic thanks, in particular, to the implementation of such a meshed blanket 7A, 7A′.

Advantageously, said barrier layer 5, 5′ and, also preferably said adhesive sub-layer 6, 6′, could advantageously form layers of surface area (area) greater than the respective surface areas of said primary 2, 2′, secondary 3, 3′, intermediate 4, 4′ layers and transfer layer 7, 7′. This signifies that, said layers 3, 4, 5, 7, 3′, 4′, 5′, 7′ and sub-layer 6, 6′ being assembled by stacking to form said medical device 1, 1′, the respective edges of said barrier layer 5, 5′ and adhesive sub-layer 6, 6′ will extend beyond the respective edges of the primary 2, 2′, secondary 3, 3′, intermediate 4, 4′ layers and transfer layer 7, 7′. In this way, it will be advantageously possible to place and to maintain firmly in position said medical device 1, 1′ against the injured skin or mucous membranes, through the respective edges of said barrier layer 5, 5′ and adhesive sub-layer 6, 6′, said transfer layer 7, 7′ coming into direct surface contact with the injured skin or mucous membrane.

Since said first zone 3A, 3A′ (or even also and advantageously said third zone 3A″) advantageously at least mainly faces said first impermeable portion 4A, 4A′, such that said intermediate layer 4, 4′ makes at least half of the surface of the first zone 3A, 3A′ (or even also advantageously said third zone 3A″) inaccessible to said liquid along a direction orthogonal to the secondary 3, 3′ and intermediate 4, 4′ layers, it is therefore readily understandable that said biosensor will be itself mainly (or even totally) inaccessible to said liquid along a direction orthogonal to the secondary 3, 3′ and intermediate 4, 4′ layers. Consequently, the risk of reflux (and in particular of “direct” reflux) of said liquid from the point of said first zone 3A, 3A′ (or even also advantageously said third zone 3A″) at the level of which is positioned said biosensor 3E, 3E′ (or even also advantageously 3E″), to the primary layer 2, 2′, is particularly low. This makes it possible to ensure the optimal operation of said biosensor. In particular, when said biosensor comprises a chemical substance potentially soluble in said liquid, the elution and the dispersion of said substance in the direction of said primary layer 2, 2′, or even to the skin or the mucous membranes in contact with which the medical device 1, 1′ is placed, is thus avoided at least in part.

Advantageously, said medical device 1, 1′ could also comprise an additional intercalary layer (not illustrated) which will be interposed between the intermediate 4, 4′ and secondary 3, 3′ layers, and more precisely at least between said first zone 3A, 3A′ and said first impermeable portion 4A, 4A′ (or even also alternatively or preferably at least between said third zone 3A″ and said third impermeable portion 4A″). Preferably, this additional intercalary layer will be formed of a thin film made of plastic material of which the face oriented facing the secondary layer 3, 3′ will be preferentially glued with an adhesive material not capable of reacting with said composition of agglomerated powders described previously. It will make it possible to further limit the risk of “direct” reflux of the liquid, while opposing the migration of said reactive substance to the intermediate layer 4, 4′.

The sub-layer 6, 6′ could optionally be designed and configured to extend only facing the second zone 3B, 3B′. Thus any potential risk of perturbation of the operation of said biosensor by the material of the sub-layer 6, 6′ will be avoided.

This being described, the preferential operation of the multilayer medical device 1, 1′ of the invention will now be described succinctly, with reference to the preferential embodiments illustrated in FIGS. 2, 3 and 6, in such a way as to clearly reveal the path (illustrated in the figures by arrows) taken by said liquid through the different layers of said medical device 1, 1′ during its guiding in the latter.

Firstly, a given liquid is placed in contact with the transfer layer 7, 7′. The latter being permeable to said liquid, said liquid progresses along a mean direction substantially orthogonal to said transfer layer 7, 7′ (that is to say preferably along the axis A-A′, B-B′ of FIGS. 2, 3 and 6) until reaching the primary layer 2, 2′, which is positioned above and directly in line with said transfer layer 7, 7′, in direct contact with the latter. There, said liquid is then advantageously directed, along a direction substantially parallel to the primary plane of extension of the primary layer 2, 2′, thanks to the barrier elements 2C, 2C′, to the preferential collection zone(s) 2B, 2B′, 2B″ of the primary layer 2, 2′. Then, said liquid passes through the intermediate layer 4, 4′, which is laid out above and directly in line with said primary layer 1, 1′ and in direct contact with the latter, via the permeable portion(s) 4B, 4B′, 4B″ of the intermediate layer 4, 4′, along a mean direction substantially orthogonal to said intermediate layer 4, 4′, until reaching the secondary layer 3, 3′, preferably at the level of the junction of said first 3A, 3A′ and second 3B, 3B′ zones (or even also advantageously at the level of the junction of said third 3A″ and second 3B′ zones) of the latter. Finally, once said secondary layer 3, 3′ has been reached, said liquid is displaced primarily and mainly by volume along a direction substantially parallel to the secondary plane of extension of the secondary layer 3, 3′ to said first zone 3A, 3A′ (or even also advantageously the third zone 3A″). Optionally, said liquid is then displaced, in particular, from the contour of the first zone 3A, 3A′ to said biosensor 3E, 3E′ (or even also advantageously from the contour of the third zone 3A″ to said biosensor 3E″).

As said first zone 3A, 3A′ (or even also advantageously said third zone 3A″) absorbs said liquid, the liquid which continues to flow coming from the primary layer 2, 2′ reaches the secondary layer 3, 3′ and is then displaced therein mainly by volume along a direction substantially parallel to the secondary plane of extension of the secondary layer 3, 3′ to said second zone 3B, 3B′. This majority displacement of the liquid from the primary layer 2 to the first zone 3A, 3A′ (or even also advantageously to the third zone 3A″) then to the second zone 3B, 3B′ of the secondary layer 3, 3′ is advantageously facilitated by the difference in respective capacity and rate of absorption of said primary layer 2, 2′, first zone 3A, 3A′ and second zone 3B, 3B′. On the other hand, the reflux, and in particular the “direct” reflux, that is to say along a mean direction substantially orthogonal to the primary 2, 2′ and secondary 3, 3′ layers, of said liquid from said first zone 3A, 3A′ (or even also advantageously from the third zone 3A″) of the secondary layer 3 is greatly limited due to the particular construction of said aforementioned medical device 1, 1′.

It will further be noted that, when said first 3A, 3A′ (or even also advantageously said third zone 3A″) and second 3B, 3B′ zones are advantageously chosen contiguous, and said second zone 3B, 3B′ surrounding said first zone 3A, 3A′ (or even also advantageously said third zone 3A″), the presence of a greater quantity of liquid in said second zone 3B, 3B′ than in said first zone 3A, 3A′ (or even also advantageously in said third zone 3A″) also tends to prevent (or in any case, to greatly limit) any displacement of said liquid from the first zone 3A, 3A′ (or even also advantageously from said third zone 3A″) to the second zone 3B, 3B′.

Manufacturing Method

The invention also relates to, as such, a method for manufacturing a medical device 1, 1′, which medical device is advantageously pursuant to the description that has been made thereof above. Consequently, the description that precedes in relation with the medical device 1, 1′ also applies to the present manufacturing method.

In the method of the invention, a biosensor is thus produced comprising a piece made of absorbent, hydrophilic material—having a preferentially fibrous structure—fixing, on the surface and/or within the thickness thereof, a composition of agglomerated powders comprising particles of EVA having a surface in part covered by at least one orthophosphoric acid salt and a visual indicator of microbiological growth, such as described previously.

The particles of EVA are advantageously incorporated in the piece made of absorbent, hydrophilic material in the form of a composition of agglomerated powders.

The method according to the invention thus comprises a step of incorporation in a piece made of absorbent, hydrophilic material of a composition of agglomerated powders comprising particles of EVA having a surface in part covered with at least one orthophosphoric acid salt and a visual indicator of microbiological growth.

The incorporation of a dry composition within the thickness of a fibrous substrate may be carried out in various ways, notably by so-called dry impregnation techniques. To this end, a dry and powdery composition is sprinkled on the surface of a porous substrate, then the particles of said composition are made to vibrate, under the action of ultrasonic waves (cf. for example, FR 2 866 578) or instead under the action of an alternating electric field (cf. for example, WO 2015/044605, WO 2010/001043 or WO 99/22920). These particles penetrate and then progressively sink into the cavities of the porous body.

The piece made of absorbent, hydrophilic material being delimited by a closed outer contour, said composition of agglomerated powders of the biosensor will be advantageously positioned at a distance from said outer contour, preferably substantially at the centre of said piece made of absorbent, hydrophilic material.

For the manufacture of a biosensor according to the invention, dry impregnation techniques implementing an alternating electric field have proven to be particularly suited to enabling the incorporation of a powder within the thickness of an absorbent, hydrophilic material, having a fibrous structure. Hence the technical teaching dispensed by WO 2015/044605, WO 2010/001043 and WO 99/22920 form an integral part of the present description.

Advantageously, the quantity of composition of agglomerated powders incorporated in the biosensor according to the invention is equivalent to a concentration comprised between 10 mg·cm⁻³ and 100 mg·cm⁻³, preferentially between 30 mg·cm⁻³ and 50 g·cm⁻³.

Once the dry impregnation has been carried out, a thermal treatment makes it possible to make the EVA sticky and to fix the particles of agglomerated powders to the fibres of the absorbent substrate. This thermal treatment advantageously consists in an operation of calendering. Calendering, by the applied pressure and the heating temperature, makes it possible to fix and to retain durably, within the thickness of the piece made of absorbent, hydrophilic material, the particles of EVA with the visual indicator(s) of microbiological growth exposed at their surface. Calendering also improves the planeness of the surface of the piece made of absorbent, hydrophilic material and enhances its capillarity and absorption capacities.

The method according to the invention thus advantageously comprises a step of thermal treatment, advantageously of calendering, of a piece made of absorbent, hydrophilic material incorporating the composition of agglomerated powders described previously. The thermal treatment, advantageously the calendering, is (are) carried out advantageously at a temperature comprised between 50° C. and 80° C.

In a preferred embodiment, the medical device 1, 1′ further comprises at least one primary layer 2, 2′ intended to come, preferably primarily, into contact with said liquid.

Preferably, said method comprises a step of supplying or manufacturing said primary layer 2, 2′, which is preferentially formed of a piece of textile 2A, 2A′, non-woven, porous and/or hydrophilic. Advantageously in one piece, and preferentially constituted of 80% wood pulp (cellulose) and 20% polyester fibres (for example PET), such a primary piece 2A, 2A′ of textile may, for example, be obtained by wet or paper making technique (“wet laid” method), by incorporation of polyester fibres in a cellulose paste of wood pulp.

According to the invention, said method comprises a step of producing means for guiding 2C, 2C′ said liquid to at least one predefined preferential collection zone 2B, 2B′, 2B″ of said liquid (identified in dotted lines in FIGS. 2, 3 and 6), from said primary layer 2, 2′, such as has been described above. Preferably, as illustrated in the figures, said preferential collection zone(s) 2B, 2B′, 2B″ is (are) chosen substantially located at a distance from the primary edges defining the contour of said primary layer 2, 2′, so as to avoid any risk of leakage of said liquid out of the primary layer 2, 2′ through the primary edges of the latter. Preferably, said guiding means 2C, 2C′ are constituted of barrier elements 2C, 2C′ made of hydrophobic polymer material, which are advantageously in the form of longitudinal “strips”. Said production operation could then in this case be advantageously carried out by (localised) hotmelt coating of said primary piece 2A, 2A′ of textile with a hydrophobic hotmelt polymer material, for example of pressure sensitive adhesive (PSA) type. Such a coating may be implemented, for example, using at least one nozzle (or similar means) which selectively deposits said hydrophobic polymer material, in the molten state, on the surface of said primary piece 2A, 2A′ of textile. Once said molten hydrophobic polymer material has been deposited on the primary piece 2A, 2A′ of textile, it will advantageously be left to impregnate all or part of the thickness, and preferably all the thickness, of said primary piece 2A, 2A′ of textile, before it cools, sets and forms said longitudinal “strips”.

Said medical device, produced according to the method of the invention, also comprises at least:
a secondary layer 3, 3′, produced in one piece or not, and which itself comprises first 3A, 3A′ and second 3B, 3B′ zones, said first 3A, 3A′ (or even also advantageously third 3A″) and second 3B, 3B′ zones being distinct and each having a different respective behaviour in contact with said liquid;
advantageously, an intermediate layer 4, 4′, which is interposed between said primary 2, 2′ and secondary 3, 3′ layers, and which itself comprises at least one first portion impermeable 4A, 4A′ to said liquid and at least one first permeable portion 4B, 4B′ enabling the passage of said liquid coming from the primary layer 2, 2′ to the secondary layer 3, 3′, said first impermeable portion 4A, 4A′ extending facing said first zone 3A, 3A′, the projection of the first impermeable portion 4A, 4A′ in a plane parallel to said secondary 3, 3′ and intermediate 4, 4′ layers covering at least half of the surface of the projection of the first zone 3A, 3A′ in this same plane, to limit the reflux of said liquid from said first zone 3A, 3A′ to said primary layer 2, 2′, and
advantageously, a barrier layer 7, 7′, covering said secondary layer 3, 3′ and being substantially impermeable to said liquid.

Advantageously conforming to the description that has been respectively made above in relation with the medical device 1, 1′ of the invention, said primary 2, 2′, secondary 3, 3′, intermediate 4, 4′ and barrier 7, 7′ layers are preferentially distinct from each other. In particular, said primary layer 2, 2′ is preferentially produced from a primary piece 2A, 2A′ of textile, which textile is preferentially non-woven and hydrophilic, said primary piece 2A, 2A′ of textile being advantageously draining.

Preferably, the method of the invention comprises an operation of manufacture of said intermediate layer 4, 4′, by coating of the primary layer 2, 2′ or the secondary layer 3, 3′ with a hydrophobic polymer material. This hydrophobic polymer material could, for example, be a pressure sensitive adhesive or polyurethane. In a preferential manner, this operation of manufacture of the intermediate layer 4, 4′ will be carried out by partial coating of a face of one or the other of said primary 2, 2′ and secondary 3, 3′ layers, in such a way as to produce at least one coated portion, forming said first impermeable portion 4A, 4A′, and at least one non-coated portion, forming for its part said first permeable portion 4B, 4B′ of the intermediate layer 4, 4′. Depending on the nature of said hydrophobic polymer material retained, said operation of manufacture of the intermediate layer 4, 4′ could be carried out by hotmelt coating, optionally followed by cross-linking (by IR, UV or other radiation), or instead by transfer.

In a particularly advantageous manner, apart from the calendering described previously for the production of the biosensor, the method of the invention could comprise an operation of compression of the composition of agglomerated powders of the biosensor 3E, 3E′ and of said first zone 3A, 3A′ of the secondary layer 3, 3′. Preferably, said compression operation is carried out hot, under conditions of controlled pressure and temperature to limit notably the risks of degradation of the properties of the composition of agglomerated powders (for example, at a temperature less than or equal to 80° C.). Preferably, said compression operation is an operation of localised compression of said biosensor 3E, 3E′ and of said first zone 3A, 3A′ of the secondary layer 3, 3′, at the level of the zone 3F, 3F′ comprising said agglomerated composition, that is to say that only a part of the latter is subjected to a compressive force, for example along a rectilinear compression line 8 passing through said zone 3F, 3F′, such as delimited by dashes in FIG. 7. Preferably, said compression operation will be carried out before the assembly of the secondary layer 3, 3′ relative to the first primary layer 2, 2′. According to a preferential embodiment of the method according to the invention, said compression operation is advantageously implemented using a heating plate of which the surface is provided with a zone in relief, and which is pressed against the first zone 3A, 3A′ and the zone 3F, 3F′ of the secondary layer 3, 3′, so as to cause local crushing of material.

Such a compression operation advantageously makes it possible to favour, by capillarity, the placing in contact of the liquid arriving at the level of the first zone 3A, 3A′ with the composition of agglomerated powders of the biosensor 3E, 3E′.

In a more general manner, it could be interesting to create one or more points of compression of all or part of the layers of the medical device 1, 1′ between them, in order to favour the displacement of the liquid within the latter, according to a principle similar to that described above.

### EXAMPLES

### Example 1

### Demonstration of the Bacteriostatic Effect of EVA and the Inhibition of this Effect by Addition of Orthophosphoric Acid Salts (KH₂PO₄, NaH₂PO₄)

### A. Demonstration Carried Out on Agar Culture Media

The effects of EVA and orthophosphoric acid salts (more particularly, KH₂PO₄ and/or NaH₂PO₄) on bacterial growth and development were evaluated, first of all, on agar culture media, in this particular case:
a Trypto-Casein-Soy Agar (TSA), a universal medium known as being able to be suited to the culture and to the enrichment of most bacteria, whether they are aerobic or anaerobic;
SAID® agar (bioMérieux, France), a culture medium sold for the specific detection of S. aureus,

To this end, strains of S. aureus originating from the collection of the Applicant were used. Before inoculating them with the aforementioned two agars, the bacteria (100 mL of a suspension of 10³ CFU·mL⁻¹) were resuspended beforehand in human serum prepared and supplied by the Etablissement Francais du Sang (EFS), complemented or not with EVA and/or with orthophosphoric acid salt as follows:
s01: serum (EFS, reference 4566200/4566201)
s02: serum s01 with addition of EVA (at 53 g·L⁻¹, of final concentration)
s03: serum s01 with addition of EVA and NaH₂PO₄ (respectively at 53 g·L⁻¹ and 4.5 g·L⁻¹, of final concentrations)
s04: serum s01 with addition of EVA and NaH₂PO₄ (respectively at 53 g·L-1 and 9.0 g·L⁻¹, of final concentrations)
s05: serum s01 with addition of NaH₂PO₄ (at 4.5 g·L⁻¹, of final concentration)
s06: serum s01 with addition of NaH₂PO₄ (at 9.0 g·L⁻¹, of final concentration)
s07: serum s01 with addition of EVA and KH₂PO₄ (respectively at 53 g·L⁻¹ and 10.6 g·L⁻¹, of final concentrations)
s08: serum s01 with addition of EVA and KH₂PO₄ (respectively at 53 g·L⁻¹ and 1.5 g·L⁻¹, of final concentrations)
s09: serum s01 with addition of EVA and KH₂PO₄ (respectively at 53 g·L⁻¹ and 3.0 g·L⁻¹, of final concentrations)
s10: serum s01 with addition of KH₂PO₄ (at 1.5 g·L⁻¹, of final concentration)
s11: serum s01 with addition of EVA, NaH₂PO₄ and KH₂PO₄ (respectively at 53 g·L⁻¹, 4.5 g·L⁻¹ and 1.5 g·L⁻¹, of final concentrations)
s12: serum s01 with addition of EVA, NaH₂PO₄ and KH₂PO₄ (respectively at 53 g·L⁻¹, 9.0 g·L⁻¹ and 3.0 g·L⁻¹, of final concentrations)
s13: serum s01 with addition of NaH₂PO₄ and KH₂PO₄ (respectively at 4.5 g·L⁻¹ and 1.5 g·L⁻¹, of final concentrations)
s14: serum s01 with addition of NaH₂PO₄ and KH₂PO₄ (respectively at 9.0 g·L⁻¹ and 3.0 g·L⁻¹, of final concentrations).

The EVA used in these tests is sold by the DAKOTA Company, Belgium, in the form of a reference powder UNEX EVA (EVA Ti). Its vinyl acetate content by weight is 28%.

The results obtained are compiled in Table 1, hereafter.

TABLE 1

| Serums | TSA | SAID |
|---|---|---|
| s01 | bacterial lawn | bacterial lawn |
| s02 | 1 colony | nothing |
| s03 | >300 colonies | >300 colonies |
| s04 | 300 colonies | 300 colonies |
| s05 | bacterial lawn | bacterial lawn |
| s06 | bacterial lawn | bacterial lawn |
| s07 | nothing | nothing |
| s08 | bacterial lawn | bacterial lawn |
| s09 | bacterial lawn | bacterial lawn |
| s10 | bacterial lawn | bacterial lawn |
| s11 | nothing | nothing |
| s12 | nothing | nothing |
| s13 | bacterial lawn | bacterial lawn |
| s14 | bacterial lawn | bacterial lawn |

### B. Demonstration Carried Out on an Absorbent and Hydrophilic Culture Support

The evaluation of the effects of EVA and orthophosphoric acid salts (more particularly, KH₂PO₄) on bacterial growth and development was continued on an absorbent and hydrophilic culture support, incorporating EVA and imbibed with a nutritive solution comprising, among others, an orthophosphoric acid salt.

1. Preparation of the Absorbent and Hydrophilic Culture Support

The absorbent and hydrophilic culture support used is here a piece of Airlaid® originating from the SCA Company, France (reference 95NN81) and having an announced grammage of 95 g·m⁻², for a thickness of 2 mm.

Within the thickness of this material made of cellulose fibres were immobilised particles of EVA of which the surface was covered with a powdery composition comprising a chromogenic substrate (for example 5-bromo-4-chloro-3-indolyl-α-glucopyranoside, 5-bromo-4-chloro-3-indolyl-N-methyl-α-glucopyranoside), xanthan gum and an agent for stimulating the metabolism of bacteria (MnCl₂).

Xanthan gum is used to increase slightly the absorption capacity of the Airlaid® and to generate a gel that will facilitate the implantation and the retention of bacteria within the thickness of the fibrous material. MnCl₂ for its part makes it possible to stimulate bacterial metabolism. 5-bromo-4-chloro-3-indolyl-α-glucopyranoside and 5-bromo-4-chloro-3-indolyl-N-methyl-α-glucopyranoside, under the action of hydrolysis of α-glucosidase, release chromophores of blue/green colour visible to the naked eye. These chromogenic substrates thus make it possible to mark bacterial colonies expressing α-glucosidase activity which could develop on this culture support, such as for example colonies of S. aureus.

More precisely, the tests were carried out with square pieces of Airlaid®, with 2.7 cm sides. These pieces made of cellulose fibres were dry impregnated with a composition of powders comprising (for around 100 g of composition):
80 g of EVA, with an average particle size of the order of 60-80 μm.
20 g of xanthan gum, with an average particle size of the order of 20-30 μm.
0.533 g of 5-bromo-4-chloro-3-indolyl-N-methyl-α-glucopyranoside and 0.3 g of 5-bromo-4-chloro-3-indolyl-α-glucopyranoside, with an average particle size of the order of 20-30 μm,
0.027 mg of MnCl₂, with an average particle size of the order of 20-30 μm.

To facilitate the handling of this powder composition, in particular to obtain good homogeneous distribution of each constituent in the absorbent support, it was subjected beforehand to a hot mixing method with a view to agglomerating the particles to each other.

Thanks to heating and appropriate stirring, the surface of the particles of EVA was rendered sticky and the particles of the other constituents of the composition of powders adhere thereto. The heating was carried out at a temperature of the order of 50-60° C., which leads to the softening of the particles of EVA and makes their surface sticky. Mixing was carried out in a mechanical manner.

This power composition thus hot assembled contains particles of EVA, more or less clearly individualised, of which the surface is in part lined with particles of 5-bromo-4-chloro-3-indolyl-N-methyl-α-glucopyranoside and 5-bromo-4-chloro-3-indolyl-α-glucopyranoside, particles of xanthan gum and particles of MnCl₂. It was next transferred within the thickness of a sheet of Airlaid® by a dry impregnation technique such as for example described in WO 2015/044605, WO 2010/001043 and WO 99/22920. This technique consists in sprinkling the sheet of Airlaid® with the powder composition and vibrating the particles of said composition under the action of an alternating electric field; the particles penetrate and then progressively sink into the cavities of the fibrous body.

Once impregnated, the sheet of Airlaid® was calendered between two sheets of greaseproof paper, the impregnated face being turned facing the heating plate. This calendering was carried out under 4 bars of pressure and at 80° C., for 1-2 minutes.

The sheet of Airlaid® was cut into pieces of 2.7 cm sides. Each of these squares of Airlaid® contained around 0.06 g of powder, containing around 80% of EVA.

2. Bacterial Strains Used and Preparation of the Nutritive Solution

Strains of Staphylococcus aureus were used, in this particular case a methicillin sensitive strain (MSSA) originating from the collection of the Applicant. Within the scope of this test, the nutrients necessary for the growth and the development of these bacteria are provided by human serum (originating from EFS) diluted with PBS. This nutritive solution aims to reproduce the nutritional qualities of the exudates of wounds.

Various compositions of synthetic or reconstituted exudates are described in the scientific literature and may be used to reproduce the test conducted by the inventors. A culture broth of low nutritional qualities or instead peptone water may also be used for this same purpose.

To evaluate the effect of the concentration of KH₂PO₄ on the growth and the development of bacteria subjected to the presence of EVA (contained here within the thickness of the piece of Airlaid®), to the serum diluted with PBS is added KH₂PO₄: 0 g·L⁻¹, 3 g·L⁻¹, 4 g·L⁻¹, 5 g·L⁻¹ and 6 g·L⁻¹ (in final concentrations).

3. Implementation of the Test and Results Obtained

100 μL of a cellular preparation concentrated to 10⁶ CFU·mL⁻¹ (in Tryptone salt) were added to 900 μL of serum diluted with PBS, with or without KH₂PO₄. The pieces of Airlaid® were soaked in this solution then incubated in a jar for 17 hours at 32° C., with a little water in the jar to avoid desiccation.

After incubation, the pieces of culture support were examined and the following observations were able to be made:
The pieces of Airlaid® seeded with bacteria and a serum diluted with PBS, exempt of KH₂PO₄, were colourless. No colony had grown thereon.
A green coloured shade was observed on the pieces seeded with bacteria and a serum diluted with PBS complemented with KH₂PO₄. A particularly marked coloration was observed for concentrations of KH₂PO₄ of 3 g·L⁻¹ and 5 g·L⁻¹. This coloration was even more intense at 4 g·L⁻¹ of KH₂PO₄. At 6 g·L⁻¹ of KH₂PO₄, it was hardly observable.

### C. Demonstration in Liquid Culture Medium

The evaluation of the effects of EVA and orthophosphoric acid salts on bacterial growth and development was also carried out in liquid medium. The objective is to evaluate more particularly the impact of EVA and KH₂PO₄ on the bacterial growth kinetics.

To this end, 1 mL of a suspension of Staphylococcus aureus (300 CFU·mL⁻¹) originating from the collection of the Applicant was cultivated at 37° C. in 9 mL of different nutritive solutions:
serum diluted with PBS,
serum diluted with PBS, and comprising 18.5 g·L⁻¹ of EVA,
serum diluted with PBS, and comprising 18.5 g·L⁻¹ of EVA and 4 g·L⁻¹ of KH₂PO₄.

After each hour elapsed, the cells were counted for a tube of culture of each series. To do so, the cells of each tube were sub-cultured on a dish of agar ChromID® S. aureus (bioMérieux, France). The dishes were incubated for 24 hours at 37° C., before counting the colonies formed.

The three growth kinetics thus measured are presented in the form of curves in FIG. 8.

These results confirm those obtained previously, on a solid culture medium. EVA has a bacteriostatic effect. This bacteriostatic effect may be inhibited by orthophosphoric acid salts, such as KH₂PO₄. Using KH₂PO₄, in a quantity by weight corresponding to the order of ⅕ of the quantity of EVA, makes it possible to counterbalance efficiently the bacteriostatic effect of EVA.

### Example 2

### Preparation of Biosensors According to the Invention and Evaluation of the Capacity Thereof for Detecting and Identifying Bacteria

A. Preparation and Assembly

The following example illustrates the production of a biosensor according to the invention, in accordance with a particular embodiment.

In this particular embodiment, said biosensor is prepared from a piece made of absorbent, hydrophilic fibrous material. Within the thickness of this material and fixed to the fibres thereof, EVA particles retain and expose on their surface a powdery mixture associating:
an orthophosphoric acid salt (KH₂PO₄),
two chromogenic substrates of α-glucosidase (5-bromo-4-chloro-3-indolyl-N-methyl-α-glucopyranoside and 5-bromo-4-chloro-3-indolyl-α-glucopyranoside)
a gelling agent (xanthan gum), and
a bacterial metabolism activator (MnCl₂).

The particles of EVA coated with said powdery mixture were prepared beforehand by hot mixing a powdery composition of dissociated particles, comprising (for around 100 g of composition):
80 g of EVA,
20 g of xanthan gum
6.67 g of KH₂PO₄,
0.533 g of 5-bromo-4-chloro-3-indolyl-N-methyl-α-glucopyranoside and 0.3 g of 5-bromo-4-chloro-3-indolyl-α-D-glucopyranoside, and
0.027 g of MnCl₂.

This powdery mixture was subjected to a method of hot mixing with a view to obtaining a composition of agglomerated powders, implemented in the same conditions as those of the hot mixing described previously.

FIG. 9 shows two photographs taken by scanning electron microscope of the powdery composition of dissociated particles, before hot mixing.

FIG. 10 shows two photographs taken by scanning electron microscope of a composition of agglomerated powders according to the invention, in which may clearly be distinguished particles of EVA, on the surface of which are spread out the finest particles, in this case particles of KH₂PO₄, 5-bromo-4-chloro-3-indolyl-N-methyl-α-glucopyranoside and 5-bromo-4-chloro-3-indolyl-α-glucopyranoside, xanthan gum and MnCl₂.

The composition of agglomerated powders thus prepared was next transferred within the thickness of a sheet of Airlaid® by a dry impregnation technique, as detailed previously.

Once impregnated, the sheet of Airlaid® was calendered between two sheets of greaseproof paper, the impregnated face being turned facing the heating plate. This calendering was carried out under 4 bars of pressure and at 80° C., for around 1 minute and 30 seconds.

Biosensors according to the invention were cut out from this sheet of Airlaid® dry impregnated with the composition of agglomerated powders, then calendered. They enable the detection of bacteria expressing α-glucosidase activity, as is the case notably for the bacteria Staphylococcus aureus.

Other biosensors of very similar design to the first example were also assembled. In these biosensors according to the invention, the particles of EVA also expose on their surface cefoxitin, an antibiotic in respect of which the strains of Staphylococcus aureus sensitive to methicillin (MSSA) are particularly sensitive, unlike methicillin resistant strains (MRSA). This second example of biosensors according to the invention enables the detection and identification of strains of MRSA.

The cefoxitin is fixed on the surface of the particles of EVA in a concomitant manner with KH₂PO₄, 5-bromo-4-chloro-3-indolyl-N-methyl-α-glucopyranoside and/or 5-bromo-4-chloro-3-indolyl-α-glucopyranoside, xanthan gum and MnCl₂, by a hot mixing method. To do so, the powdery composition of dissociated particles set out previously also comprises 6.6 mg of cefoxitin.

B. Evaluation of the Preceding Biosensors for the Detection and Identification of Staphylococcus aureus

Biosensors such as described previously and embedding a composition of agglomerated powders according to the invention, comprising particles of EVA on the surface of which are distributed:
KH₂PO₄,
5-bromo-4-chloro-3-indolyl-N-methyl-α-glucopyranoside
5-bromo-4-chloro-3-indolyl-α-glucopyranoside,
xanthan gum and
MnCl₂,

were tested for their aptitude for the detection of strains of Staphylococcus aureus, in this particular case methicillin sensitive strains (MSSA) originating from the collection of the Applicant.

To do so, on squares of biosensors, of 2.7 cm sides, was deposited 1 mL of serum diluted with PBS, comprising 10⁵ CFU of a strain of MSSA (or 1 mL of serum diluted with PBS exempt of bacteria, for the control biosensors).

The pieces of Airlaid® were then incubated in a jar for 17 hours at 32° C., with a little water in the jar to avoid desiccation.

After incubation, the biosensors were examined and the following observations were able to be made:
The control biosensors seeded uniquely with serum diluted with PBS remain colourless.
A green coloured shade was observed on the biosensors without cefoxitin, seeded with MSSA bacteria and the serum diluted with PBS.
The biosensors containing cefoxitin and seeded with MSSA bacteria and the serum diluted with PBS, remain colourless.

1. Non-implantable medical device (1, 1′) intended to cover wounds and skin lesions, comprising a biosensor (3E, 3E′), wherein said biosensor (3E, 3E′) comprises a piece made of absorbent and hydrophilic material, the material fixing a composition of agglomerated powders comprising particles of ethylene vinyl acetate (EVA) having a surface in part covered with at least one orthophosphoric acid salt and a visual indicator of microbiological growth.

2. Medical device (1, 1′) according to claim 1, wherein the orthophosphoric acid salt is chosen from potassium dihydrogen phosphate (KH₂PO₄) and sodium dihydrogen phosphate (NaH₂PO₄).

3. Medical device (1, 1′) according to claim 1, wherein the EVA and KH₂PO₄ are present in a KH₂PO₄/EVA ratio by weight comprised between 1:25 and 1:8.

4. Medical device (1, 1′) according to claim 1, wherein the EVA and NaH₂PO₄ are present in a NaH₂PO₄/EVA ratio by weight comprised between 1:15 and 1:3.

5. Medical device (1, 1′) according to claim 1, wherein the EVA has a content by weight of vinyl acetate of 10 to 40%.

6. Medical device (1, 1′) according to claim 1 further comprising a primary layer (2, 2′) suited to directing a liquid to the biosensor (3E, 3E′).

7. Medical device (1, 1′) according to claim 6, wherein the primary layer (2, 2′) has a preferential collection zone (2B, 2B′) of said liquid as well as means for guiding (2C, 2C′) said liquid in the direction of said preferential collection zone (2B, 2B′).

8. Medical device (1, 1′) according to claim 7, further comprising a secondary layer (3, 3′) integrating said biosensor (3E, 3E′), the secondary layer (3, 3′) being arranged with respect to the primary layer (2, 2′) so that said biosensor (3E, 3E′) extends facing the preferential collection zone (2B, 2B′).

9. Medical device (1, 1′) according to claim 8, wherein the secondary layer (3, 3′) comprises first (3A, 3A′) and second (3B, 3B′) zones, said first (3A, 3A′) and second (3B, 3B′) zones being distinct and each having a different respective behaviour in contact with said liquid.

10. Medical device (1, 1′) according to claim 8 further comprising an intermediate layer (4, 4′), which is interposed between said primary (2, 2′) and secondary (3, 3′) layers.

11. Medical device (1, 1′) according to claim 10, wherein the intermediate layer (4, 4′) itself comprises at least one first portion impermeable (4A, 4A′) to said liquid and at least one first permeable portion (4B, 4B′) enabling the passage of said liquid coming from the primary layer (2, 2′) to the secondary layer (3, 3′), said first impermeable portion (4A, 4A′) extending facing said first zone (3A, 3A′), the projection of the first impermeable portion (4A, 4A′) in a plane parallel to said secondary (3, 3′) and intermediate (4, 4′) layers covering at least half of the surface of the projection of the first zone (3A, 3A′) in this same plane, to limit reflux of said liquid from said first zone (3A, 3A′) to said primary layer (2, 2′).

12. Medical device (1, 1′) according to claim 8, further comprising a barrier layer (5, 5′), covering said secondary layer (3, 3′) and being substantially impermeable to said liquid.

13. Medical device (1, 1′) according to claim 12, wherein said barrier layer (5, 5′) is permeable to air.

14. Medical device (1, 1′) according to claim 8, further comprising a transfer layer (7, 7′) permeable to said liquid, arranged facing said primary layer (2, 2′) and enabling the passage of said liquid coming from the medium external to said medical device (1, 1′) to said primary layer (2, 2′).

15. Medical device (1, 1′) according to claim 1, which is a dressing or a compress.

16. Method for manufacturing a medical device (1, 1′) according to claim 1, comprising a step of incorporating a composition of agglomerated powders in a piece made of absorbent, and hydrophilic material, the composition of agglomerated powders comprising particles of ethylene vinyl acetate (EVA) having a surface in part covered with at least one orthophosphoric acid salt and a visual indicator of microbiological growth.

17. Method according to claim 16, further comprising a step of thermal treatment, at a temperature comprised between 50° C. and 80° C., of the piece made of absorbent and hydrophilic material incorporating the composition of agglomerated powders comprising particles of ethylene vinyl acetate (EVA) having a surface in part covered with at least one orthophosphoric acid salt and a visual indicator of microbiological growth.

18. Medical device (1, 1′) according to claim 1, wherein the composition of agglomerated powders is fixed on the surface of the material.

19. Medical device (1, 1′) according to claim 1, wherein the composition of agglomerated powders is fixed within the thickness of the material.

20. Medical device (1, 1′) according to claim 13, wherein said barrier layer (5, 5′) is transparent to light.

21. Method according to claim 16, wherein the orthophosphoric acid salt is chosen from potassium dihydrogen phosphate (KH₂PO₄) and sodium dihydrogen phosphate (NaH₂PO₄).

22. Method according to claim 16, wherein the EVA and KH₂PO₄ are present in a KH₂PO₄/EVA ratio by weight comprised between 1:25 and 1:8.

23. Method according to claim 16, wherein the EVA and NaH₂PO₄ are present in a NaH₂PO₄/EVA ratio by weight comprised between 1:15 and 1:3.

24. Method according to claim 16, wherein the EVA has a content by weight of vinyl acetate of 10 to 40%.
